(19) United States
(12) Patent Application Publication
(10) Pub. No.: US 2011/0053908 A1
(43) Pub. Date: Mar. 3, 2011

(54) AZETIDINE-DERIVED COMPOUNDS, PREPARATION METHOD THEREFOR AND THERAPEUTIC USE OF SAME

(75) Inventors: Florian AUGER, Paris (FR); Patrick BERNARDELLI, Villepreux (FR); Luc EVEN, Paris (FR); Jean-Francois SABUCO, Paris (FR); Corinne TERRIER, Livry Gargan (FR)

(73) Assignee: SANOFI-AVENTIS, Paris (FR)

(21) Appl. No.: 12/869,281

(22) Filed: Aug. 26, 2010

Related U.S. Application Data
(63) Continuation of application No. PCT/FR2009/000214, filed on Feb. 27, 2009.

(30) Foreign Application Priority Data
Feb. 29, 2008 (FR) 0801117

(51) Int. Cl. A61K 31/397; C07D 403/12; C07D 205/04; A61P 25/00; A61P 29/00; A61P 13/10; A61P 1/12; A61P 9/00; A61P 1/16; A61P 19/02; A61P 3/04; A61P 3/10

(52) U.S. Cl. 514/210.19; 548/953; 514/210.01

(57) ABSTRACT

This disclosure relates to compounds of formula (I):

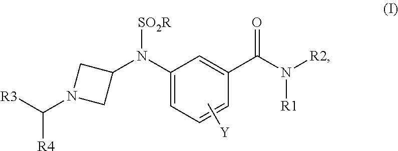

wherein R, R1, R2, R3, R4 and Y are as defined in the disclosure, or an acid addition salt thereof, and to processes for the preparation of these compounds and the therapeutic use thereof.

The present invention relates to azetidine derivatives, to their preparation and to their therapeutic application in the treatment or prevention of diseases involving CB1 cannabinoid receptors.

The subject-matter of the present invention is compounds corresponding to the formula (I)

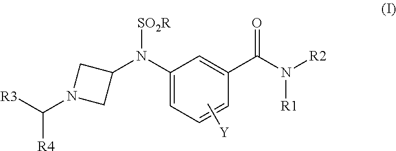

in which:
R represents a (C₁-C₆)alkyl group or a halo(C₁-C₆)alkyl group;
R1 represents a hydrogen atom or a (C₁-C₆)alkyl group;
R2 represents a
   (C₁-C₆)alkyl group substituted by one or more groups chosen from the hydroxyl group, the (C₁-C₆)alkoxy group, a hydroxy(C₁-C₆)alkyl group and optionally substituted by a halo(C₁-C₆)alkyl group;
   heterocycle group optionally substituted by one or more hydroxyls, a (C₁-C₆)alkoxy group or a hydroxy(C₁-C₆)alkyl group;
   heterocycle(C₁-C₆)alkyl group optionally substituted by one or more hydroxyls;
R3 and R4 each represent a phenyl group, optionally substituted by one or more atoms or groups chosen from a hydrogen atom, a halogen, a (C₁-C₆)alkyl group, a halo(C₁-C₆)alkyl group, a (C₁-C₆)alkoxy group, a halo(C₁-C₆)alkoxy group or cyano;
Y represents a hydrogen atom, a halogen, a (C₁-C₆)alkyl group, a halo(C₁-C₆)alkyl group, a (C₁-C₆)alkoxy group, a halo(C₁-C₆)alkoxy group, a (C₁-C₆)alkylS(O)ₚ group or cyano;
p is 0, 1 or 2;
in the form of the base or of an addition salt with an acid.

The compounds of formula (I) can comprise one or more asymmetric carbon atoms. They can thus exist in the form of enantiomers or diastereoisomers. These enantiomers or diastereoisomers and their mixtures, including racemic mixtures, come within the invention.

Among the compounds of formula (I) which are subject-matters of the invention, a first group of compounds is composed of the compounds, as a mixture of diastereoisomers and of enantiomers, for which:
R represents a methyl,
R3 and R4 each represent a phenyl group substituted by a chlorine atom in the para position,
Y represents a hydrogen atom or a halogen or a (C₁-C₆)alkoxy group or a halo(C₁-C₆)alkyl group,
R1 represents a hydrogen atom,
R2 represents a
   (C₁-C₆)alkyl group substituted by one or more groups chosen from the hydroxyl group, a (C₁-C₆)alkoxy group, a hydroxy(C₁-C₆)alkyl group and optionally substituted by a halo(C₁-C₆)alkyl group;
   heterocycle group representing an oxetane, a tetrahydrofuran, a dioxolane or a tetrahydropyran optionally substituted by one or more hydroxyls or hydroxymethyls;
   heterocycle(C₁-C₆)alkyl group representing a tetrahydrofurylmethyl, 2,2-dimethyl-1,3-dioxolan-4-ylmethyl or 1,3-dioxolan-4-ylmethyl;
      in the form of the base or of an addition salt with an acid.

Among the compounds of formula (I) which are subject-matters of the invention, a second group of compounds is composed of the compounds, as a mixture of diastereoisomers and of enantiomers, for which:
R represents a methyl,
R3 and R4 each represent a phenyl group substituted by a chlorine atom in the para position,
Y represents a hydrogen atom or a fluorine or an OMe group or a CF₃ group,
R1 represents a hydrogen atom,
R2 represents a
   (C₁-C₆)alkyl group substituted by one or more groups chosen from the hydroxyl group, the (C₁-C₆)alkoxy group, a hydroxy(C₁-C₆)alkyl group and optionally substituted by a halo(C₁-C₆)alkyl group;
   an oxetane, a tetrahydrofuran, a dioxolane or a tetrahydropyran optionally substituted by one or more hydroxyls or hydroxymethyls;
   a tetrahydrofurylmethyl, 2,2-dimethyl-1,3-dioxolan-4-ylmethyl or 1,3-dioxolan-4-ylmethyl;
      in the form of the base or of an addition salt with an acid.

The combinations of the abovementioned groups are also groups of compounds which are subject-matters of the invention.

In the context of the present invention:
a halogen is understood to mean a fluorine, a chlorine, a bromine or an iodine;
a (C₁-C₆)alkyl group is understood to mean a saturated, cyclic, branched or linear, aliphatic group comprising from 1 to 6 carbon atoms which can optionally be substituted by one or more linear, branched or cyclic (C₁-C₆)alkyl groups. Mention may be made, as examples, of the methyl, ethyl, propyl, isopropyl, butyl, isobutyl, tert-butyl, pentyl, hexyl, cyclopropyl, cyclobutyl, cyclopentyl, cyclohexyl, cycloheptyl, cyclopropylmethyl or cyclobutylmethyl groups, and the like;
a halo(C₁-C₆)alkyl group is understood to mean a (C₁-C₆)alkyl group, one or more hydrogen atoms of which have been substituted by a halogen atom. Mention may be made, as examples, of the CF₃, CH₂CF₃, CHF₂, CCl₃ groups;
a hydroxy(C₁-C₆)alkyl group is understood to mean a (C₁-C₆)alkyl group, one hydrogen atom of which has been substituted by one or more hydroxyls;
a (C₁-C₆)alkoxy group is understood to mean a (C₁-C₆)alkyl-O— group where the (C₁-C₆)alkyl group is as defined above;
a halo(C₁-C₆)alkoxy is understood to mean a halo(C₁-C₆)alkyl-O— group where the halo(C₁-C₆)alkyl group is as defined above;
a heterocycle group is understood to mean a monocyclic group comprising from 4 to 8 atoms, including 1 to 3 oxygen atoms, this cyclic group being saturated or partially saturated. Mention may be made, as examples, of the oxetane, tetrahydrofuran, dioxolane or tetrahydropyran groups;
a heterocycle(C₁-C₆)alkyl group is understood to mean an alkyl group substituted by a heterocycle as defined above. Mention may be made, as examples, of tetrahydrofuranylmethyl, 2,2-dimethyl-1,3-dioxolan-4-ylmethyl or 1,3-dioxolan-4-ylmethyl.

The compounds of formula (I) can exist in the form of bases or of salts. Such addition salts come within the invention.

These salts can be prepared with pharmaceutically acceptable acids but the salts of other acids of use, for example, in the purification or the isolation of the compounds of formula (I) also come within the invention.

The compounds of formula (I) can also exist in the form of hydrates or of solvates, namely in the form of combinations or associations with one or more molecules of water or with a solvent. Such hydrates or solvates also come within the invention.

Among the compounds of formula (I) which are subject-matters of the invention, mention may in particular be made of the following compounds; the nomenclature used corresponds to the IUPAC nomenclature;(+)-3-({1-[bis(4-chlorophenyl)methyl]azetidin-3-yl}(methanesulphonyl)amino)-N-[1(tetrahydrofuran-2-yl)methyl]benzamide

(−)-3-({1-[bis(4-chlorophenyl)methyl]azetidin-3-yl}(methanesulphonyl)amino)-N-[1-(tetrahydrofuran-2-yl)methyl]benzamide

3-({1-[bis(4-chlorophenylmethyl]azetidin-3-yl}(methanesulphonyl)amino)-N-(2,2-dimethyl-1,3-dioxolan-4-ylmethyl)benzamide

(+)-3-({1-[bis(4-chlorophenyl)methyl]azetidin-3-yl}(methanesulphonyl)amino)-N-(2,2-dimethyl-1,3-dioxolan-4-ylmethyl)benzamide

(−)-3-({1-[bis(4-chlorophenyl)methyl]azetidin-3-yl}(methanesulphonyl)amino)-N-(2,2-dimethyl-1,3-dioxolan-4-ylmethyl)benzamide

3-({1-[bis(4-chlorophenyl)methyl]azetidin-3-yl}(methanesulphonyl)amino)-N-(oxetan-3-yl)benzamide

3-({1-[bis(4-chlorophenyl)methyl]azetidin-3-yl}methanesulphonylamino)-N-(2-hydroxyethyl)benzamide hydrochloride (1:1)

(−)-3-({1-[bis(4-chlorophenyl)methyl]azetidin-3-yl}(methanesulphonyl)amino)-N-(1-hydroxyprop-2-yl)benzamide

(+)-3-({1-[bis(4-chlorophenyl)methyl]azetidin-3-yl}(methanesulphonyl)amino)-N-(1-hydroxyprop-2-yl)benzamide

(−)-3-({1-[bis(4-chlorophenyl)methyl]azetidin-3-yl}(methanesulphonyl)amino)-N-(2-hydroxyprop-1-yl)benzamide

(+)-3-({1-[bis(4-chlorophenyl)methyl]azetidin-3-yl}(methanesulphonyl)amino)-N-(2-hydroxyprop-1-yl)benzamide

3-({1-[bis(4-chlorophenyl)methyl]azetidin-3-yl}(methanesulphonyl)amino)-N-(3,3,3-trifluoro-2-hydroxyprop-1-yl)benzamide

3-({1-[bis(4-chlorophenyl)methyl]azetidin-3-yl}(methanesulphonyl) amino)-N-(2-hydroxy-2-methylprop-1-yl)benzamide

3-({1-[bis(4-chlorophenyl)methyl]azetidin-3-yl}(methanesulphonyl)amino)-N-(1-(hydroxymethyl)cyclopent-1-yl)benzamide

3-({1-[bis(4-chlorophenyl)methyl]azetidin-3-yl}(methanesulphonyl)amino)-N—((S)-1-hydroxymethyl-2-methylprop-1-yl)benzamide

3-({1-[bis(4-chlorophenyl)methyl]azetidin-3-yl}(methanesulphonyl)amino)-N-(2-hydroxy-1,1-dimethylethyl)benzamide

3-({1-[bis(4-chlorophenyl)methyl]azetidin-3-yl}(methanesulphonyl)amino)-N-(1,3-dihydroxyprop-2-yl)benzamide

3-({1-[bis(4-chlorophenyl)methyl]azetidin-3-yl}(methanesulphonyl)amino)-N-[1,3-dihydroxy-2-methylprop-2-yl]benzamide

3-({1-[bis(4-chlorophenyl)methyl]azetidin-3-yl}(methanesulphonyl)amino)-N-[2-hydroxy-1,1-bis(hydroxymethyl)ethyl]benzamide

(2R,3R,4R,5S,6R)-3-({1-[bis(4-chlorophenyl)methyl]azetidin-3-yl}(methanesulphonyl)amino)-N-[2,4,5-trihydroxy-6-(hydroxymethyl)tetrahydropyran-3-yl]benzamide

3-({1-[bis(4-chlorophenyl)methyl]azetidin-3-yl}(methanesulphonyl)amino)-N-[1-(2-hydroxyethyl)cyclopropyl]benzamide

(−)-3-({1-[bis(4-chlorophenyl)methyl]azetidin-3-yl}(methanesulphonyl)amino)-N-(2,3-dihydroxyprop-1-yl)benzamide

(+)-3-({1-[bis(4-chlorophenyl)methyl]azetidin-3-yl}(methanesulphonyl)amino)-N-(2,3-dihydroxyprop-1-yl)benzamide

3-({1-[bis(4-chlorophenyl)methyl]azetidin-3-yl}(methanesulphonyl)amino)-N-(2-methoxy-ethyl)benzamide

(+)-3-({1-[bis(4-chlorophenyl)methyl]azetidin-3-yl}(methanesulphonyl)amino)-5-fluoro-N-(1-hydroxyprop-2-yl)benzamide

3-({1-[bis(4-chlorophenyl)methyl]azetidin-3-yl}(methanesulphonyl)amino)-5-fluoro-N-(1,3-dihydroxyprop-2-yl)benzamide

3-({1-[bis(4-chlorophenyl)methyl]azetidin-3-yl}(methanesulphonyl)amino)-5-fluoro-N-[1,3-dihydroxy-2-methylprop-2-yl]benzamide

3-({1-[bis(4-chlorophenyl)methyl]azetidin-3-yl}(methanesulphonyl)amino)-N-(1-(hydroxymethyl)cycloprop-1-yl)benzamide

3-({1-[bis(4-chlorophenyl)methyl]azetidin-3-yl}(methanesulphonyl)amino)-N-[(1-(hydroxymethyl)cycloprop-1-yl)methyl]benzamide

3-({1-[bis(4-chlorophenyl)methyl]azetidin-3-yl}(methanesulphonyl)amino)-5-fluoro-N-(3,3,3-trifluoro-2-hydroxyprop-1-yl)benzamide

3-({1-[bis(4-chlorophenyl)methyl]azetidin-3-yl}(methanesulphonyl)amino)-5-fluoro-N-[1-(2-hydroxyethyl)cycloprop-1-yl]benzamide

3-({1-[bis(4-chlorophenyl)methyl]azetidin-3-yl}(methanesulphonyl)amino)-5-fluoro-N-[1-(hydroxymethyl)cycloprop-1-yl]benzamide

3-({1-[bis(4-chlorophenyl)methyl]azetidin-3-yl}(methanesulphonyl)amino)-5-fluoro-N-[(1-(hydroxymethyl)cycloprop-1-yl)methyl]benzamide

3-({1-[bis(4-chlorophenyl)methyl]azetidin-3-yl}(methanesulphonyl)amino)-5-fluoro-N-[(1-(hydroxymethyl)cyclobut-1-yl)methyl]benzamide

3-({1-[bis(4-chlorophenyl)methyl]azetidin-3-yl}(methanesulphonyl)amino)-N-(2-hydroxyethyl)-5-(trifluoromethyl)benzamide

(+)-3-({1-[bis(4-chlorophenyl)methyl]azetidin-3-yl}(methanesulphonyl)amino)-N—((S)-1-hydroxyprop-2-yl)-5-(trifluoromethyl)benzamide

3-({1-[bis(4-chlorophenyl)methyl]azetidin-3-yl}(methanesulphonyl)amino)-N-(1,3-dihydroxyprop-2-yl)-5-(trifluoromethyl)benzamide

3-({1-[bis(4-chlorophenyl)methyl]azetidin-3-yl}(methanesulphonyl)amino)-N-[1,3-dihydroxy-2-methylprop-2-yl]-5-(trifluoromethyl)benzamide

3-({1-[bis(4-chlorophenyl)methyl]azetidin-3-yl}(methanesulphonyl)amino)-N-[1-(2-hydroxyethyl)cycloprop-1-yl]-5-(trifluoromethyl)benzamide

3-({1-[bis(4-chlorophenyl)methyl]azetidin-3-yl}(methanesulphonyl)amino)-N-((1RS,2SR)-2-hydroxycyclopent-1-yl)-5-(trifluoromethyl)benzamide

3-({1-[bis(4-chlorophenyl)methyl]azetidin-3-yl} (methanesulphonyl)amino)-N-((1SR,2SR)-2-hydroxycyclopent-1-yl)-5-(trifluoromethyl)benzamide

3-({1-[bis(4-chlorophenyl)methyl]azetidin-3-yl}(methanesulphonyl)amino)-5-fluoro-N-((1RS,2SR)-2-hydroxycyclopent-1-yl)benzamide

(−)-3-({1-[bis(4-chlorophenyl)methyl]azetidin-3-yl}(methanesulphonyl)amino)-5-fluoro-N-((1R*,2S*)-2-hydroxycyclopent-1-yl)benzamide

(+)-3-({1-[bis(4-chlorophenyl)methyl]azetidin-3-yl}(methanesulphonyl)amino)-5-fluoro-N-((1S*,2R*)-2-hydroxycyclopent-1-yl)benzamide

3-({1-[bis(4-chlorophenyl)methyl]azetidin-3-yl}(methanesulphonyl)amino)-5-fluoro-N-((1SR,2SR)-2-hydroxycyclopent-1-yl)benzamide

(+)-3-({1-[bis(4-chlorophenyl)methyl]azetidin-3-yl}(methanesulphonyl)amino)-N—((S)-1-hydroxyprop-2-yl)-5-methoxybenzamide

3-({1-[bis(4-chlorophenyl)methyl]azetidin-3-yl}(methanesulphonyl)amino)-5-fluoro-N-((3SR,4RS)-4-hydroxytetrahydrofuran-3-yl)benzamide

(−)-3-({1-[bis(4-chlorophenyl)methyl]azetidin-3-yl}(methanesulphonyl)amino)-5-fluoro-N-((1S*,2S*)-2-hydroxycyclopent-1-yl)benzamide

(+)-3-({1-[bis(4-chlorophenyl)methyl]azetidin-3-yl}(methanesulphonyl)amino)-5-fluoro-N-((1R*,2R*)-2-hydroxycyclopent-1-yl)benzamide

their optical isomers and their pharmaceutically acceptable salts.

Another subject-matter of the present invention is the use of the compounds of the invention of formula (I) in the preparation of a medicament for the treatment or prevention of diseases in which the CB1 receptor is implicated.

Another subject-matter of the present invention is the use of the compounds of the invention of formula (I) in the preparation of a medicament for the treatment or prevention of psychiatric disorders, substance dependence and withdrawal, tobacco withdrawal, cognitive and attention disorders and acute and chronic neurodegenerative diseases; metabolic disorders, appetency disorders, appetite disorders, obesity, diabetes (type I and/or II), metabolic syndrome, dyslipidaemia, sleep apnoea; pain, neuropathic pain, neuropathic pain induced by anticancer drugs; gastrointestinal disorders, vomiting, ulcers, diarrhoea, bladder and urinary disorders, disorders of endocrine origin, cardiovascular disorders, hypotension, haemorrhagic shock, septic shock, liver diseases, chronic liver cirrhosis, fibrosis, non-alcoholic steatohepatitis (NASH), steatohepatitis and hepatic steatosis, whatever the etiology of these conditions (alcohol, medicament, chemical, autoimmune disease, obesity, diabetes, congenital metabolic disease); diseases of the immune system, rheumatoid arthritis, demyelination, multiple sclerosis, inflammatory diseases, Alzheimer's disease, Parkinson's disease, schizophrenia, cognitive disorders associated with schizophrenia, with diabetes, with obesity or with metabolic syndrome; asthma, chronic obstructive pulmonary disease, Raynaud's syndrome, glaucoma, fertility disorders; infectious and viral diseases, such as encephalitis, strokes, Guillain-Barré syndrome, osteoporosis and sleep apnoea and for anticancer chemotherapy; disorders related to antipsychotic treatments (weight gain, metabolic disorder).

In accordance with the invention, the compounds of general formula (I) can be prepared according to the process described in Scheme 1:

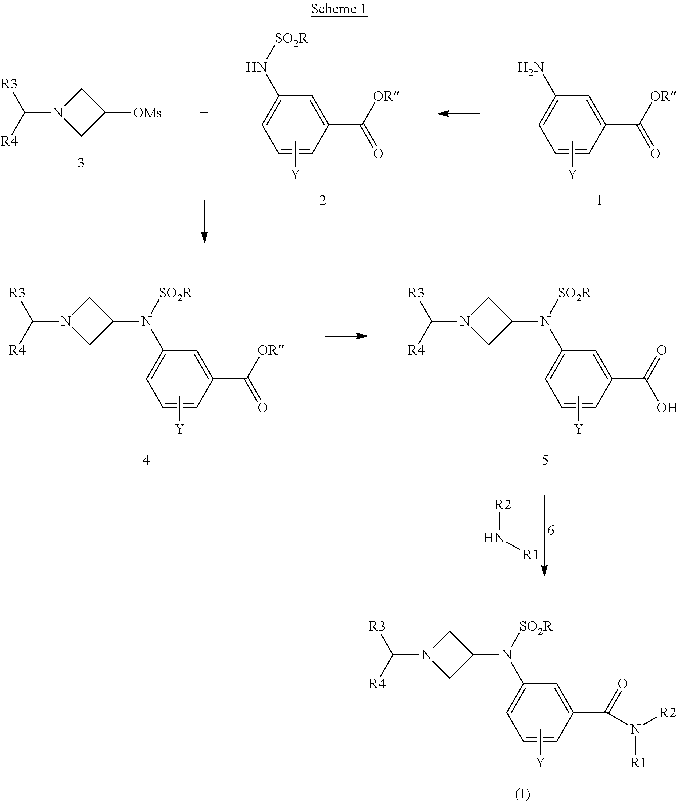

The mesylation of the compound 1 to give the derivative 2 can be carried out according to methods known to a person skilled in the art or else described in T. W. Greene, Protective Groups in Organic Synthesis, fourth edition. This reaction will be carried out in a chlorinated solvent, such as dichloromethane, in the presence of a base, such as pyridine, and of a mesylate derivative, such as mesyl chloride, at a temperature of between −10° C. and 40° C.

The derivatives 1 are commercially available or synthesized, according to methods known to a person skilled in the art, from the appropriate commercial precursors; R″ represents a protective group for the OH functional group of the acid.

The derivative 4 is accessible by reaction of the mesylate 2 with the azetidine 3. This stage is preferably carried out under an inert atmosphere, in an inert solvent, such as 4-methyl-2-pentanone, in the presence of an inorganic base, such as potassium carbonate, at reflux of the reaction mixture.

The synthesis of the azetidine 3 is described in Patent Application WO01/064634.

The hydrolysis of the ester 4 to give the acid 5 is carried out according to methods known to a person skilled in art and more specifically in a mixture of polar solvents, such as tetrahydrofuran and water, in the presence of a base, such as lithium hydroxide hydrate, at a temperature in the region of 20° C.

The compounds of formula (I) can be formed by reaction between the acid 5 and an amine derivative 6. This reaction can be carried out in an inert solvent, such as tetrahydrofuran or a chlorinated solvent (for example dichloromethane), in the presence or absence of a base, such as a trialkylamine (for example triethylamine), of a coupling agent, such as 1-(3-dimethylaminopropyl)-3-ethylcarbodiimide hydrochloride or a supported carbodiimide, in the presence or absence of an additive which prevents any racemization, such as 1-hydroxybenzotriazole and in the presence or absence of an agent which promotes peptide synthesis via the formation of a mixed anhydride, such as isobutyl chloroformate, at a temperature of between −20° C. and the boiling point of the solvent.

The derivatives 6 are commercially available or synthesized, according to methods known to a person skilled in the art, from appropriate commercial precursors.

In the case where R2 represents a (C₁-C₆)alkyl group substituted by one or more groups chosen from the hydroxyl group or the (C₁-C₆)alkoxy group, these products can be obtained from the products where R2 represents a heterocycle(C₁-C₆)alkyl group by deprotection of this group according to methods known to a person skilled in the art and more specifically in an inert solvent, such as tetrahydrofuran, in the presence of an acid, such as hydrochloric acid as a 1N solution in diethyl ether, at a temperature in the region of 20° C.

The compounds of formula (I) can be purified by the usual known methods, for example by crystallization, chromatography or extraction.

The enantiomers of the compounds of formula (I) can be obtained by resolution of the racemates, for example by chromatography on a chiral column according to Pirkle W. H. et al., Asymmetric Synthesis, vol. 1, Academic Press (1983), or by formation of salts or by synthesis from chiral precursors. The diastereoisomers can be prepared according to known conventional methods (crystallization, chromatography or from chiral precursors).

The present invention also relates to the process for the preparation of intermediates.

The following examples describe the preparation of some compounds in accordance with the invention. These examples are not limiting and serve only to illustrate the present invention. The numbers of the compounds in the examples refer to those given in the table below, in which the chemical structures and the physical properties of some compounds according to the invention are illustrated.

### EXAMPLE 1

### 3-({1-[Bis(4-chlorophenyl)methyl]azetidin-3-yl}(methanesulphonyl)amino)-N-(2,2-dimethyl-1,3-dioxolan-4-ylmethyl)benzamide (Compound No. 3)

1.52 g of 3-[{1-[bis(4-chlorophenyl)methyl]azetidin-3-yl}(methylsulphonyl)amino]benzoic acid, 30 cm³ of dichloromethane and 0.324 cm³ of 1-(2,2-dimethyl-1,3-dioxolan-4-yl)methanamine are stirred at a temperature in the region of 20° C. After addition of 4.27 g of scavenger resin (PS-carbodiimide, Argonaut, loading 1.3 mmol/g), the reaction medium is stirred overnight at a temperature in the region of 20° C. The resin is filtered off and the filtrate is concentrated to dryness on a rotary evaporator under reduced pressure (20 kPa). 1.17 g of product are obtained, which product is purified by flash chromatography on a cartridge comprising 30 g of Merck silica (particle size: 15-40 μm; eluent: dichloromethane/ethyl acetate 50/50). After concentrating the fractions under reduced pressure, 1.01 g of 3-({1-[bis(4-chlorophenyl)methyl]azetidin-3-yl}(methanesulphonyl)amino)-N-(2,2-dimethyl-1,3-dioxolan-4-ylmethyl)benzamide are obtained in the form of a white foam.
¹H NMR spectrum (400 MHz; (δ in ppm); (d₆-DMSO); referenced at 2.50 ppm): 1.26 (s, 3H), 1.32 (s, 3H), 2,69 (m, 2H), 2.96 (s, 3H), from 3.23 to 3.48 (partially masked m, 4H), 3.68 (dd, J=6.0 and 8.5 Hz, 1H), 3.99 (dd, J=6.0 and 8.5 Hz, 1H), 4.21 (m, 1H), 4.38 (s, 1H), 4.72 (m, 1H), 7.31 (d, J=9.0 Hz, 4H), 7.37 (d, J=9.0 Hz, 4H), from 7.45 to 7.54 (m, 2H), 7.78 (s, 1H), 7.83 (m, 1H), 8.67 (t, J=6.0 Hz, 1H);
Mass spectrum: ES m/z=618 (MH⁺, base peak);
Elemental analysis:
Calculated: C: 58.25% H: 5.38% N: 6.79%
Measured: C: 58.03% H: 5.27% N: 6.73%.

### EXAMPLE 2

### (−)-3-({1-[Bis(4-chlorophenyl)methyl]azetidin-3-yl}(methanesulphonyl)amino)-N-(2,2-dimethyl-1,3-dioxolan-4-ylmethyl)benzamide (Compound No. 5)

0.941 g of 3-[{1-[bis(4-chlorophenyl)methyl]azetidin-3-yl}(methylsulphonyl)amino]-N-(2,2-dimethyl-1,3-dioxolan-4-ylmethyl)benzamide is injected onto a column comprising 50 g of chiral stationary phase, Chiralcel OJ-H, 5 μm in SFC. Elution is carried out at 90 cm³ per minute with, as eluent, carbon dioxide in the supercritical state and a cosolvent composed of 10% methanol under a pressure of 125 bar. The laevorotatory enantiomer is eluted first. After concentrating the cosolvent, 0.405 g of (−)-3-({1-[bis(4-chlorophenyl)methyl]azetidin-3-yl}(methanesulphonyl)amino)-N-(2,2-dimethyl-1,3-dioxolan-4-ylmethyl)benzamide is obtained in the form of a white powder.
¹H NMR spectrum (400 MHz; (δ in ppm); (d₆-DMSO); referenced at 2.50 ppm): 1.26 (s, 3H), 1.31 (s, 3H), 2.69 (m, 2H), 2.97 (s, 3H), from 3.20 to 3.48 (partially masked m, 4H), 3.69 (dd, J=6.0 and 8.0 Hz, 1H), 3.99 (dd, J=6.0 and 8.0 Hz, 1H), 4.20 (m, 1H), 4.38 (s, 2H), 4.72 (m, 1H), 7.30 (d, J=9.0 Hz, 4H), 7.35 (d, J=9.0 Hz, 4H), from 7.43 to 7.54 (m, 2H), 7.78 (s, 1H), 7.83 (m, 1H), 8.69 (t, J=6.0 Hz, 1H);
Mass spectrum: ES m/z=618 (MH⁺, base peak);
Optical rotation: α_{D}=−4.5° (c=0.438, DMSO).

### EXAMPLE 3

### (+)-3-({1-[Bis(4-chlorophenyl)methyl]azetidin-3-yl}(methanesulphonyl)amino)-N-(2,2-dimethyl-1,3-dioxolan-4-ylmethyl)benzamide (Compound No. 4)

The dextrorotatory enantiomer was eluted in second position during the separation carried out in Example 2. After concentrating the cosolvent, 0.342 g of (+)-3-({1-[bis(4-chlorophenyl)methyl]azetidin-3-yl}(methanesulphonyl)amino)-N-(2,2-dimethyl-1,3-dioxolan-4-ylmethyl)benzamide is obtained in the form of a white powder.
¹H NMR spectrum (400 MHz; (δ in ppm); (d₆-DMSO); referenced at 2.50 ppm): 1.26 (s, 3H); 1.31 (s, 3H), 2.69 (m, 2H), 2.96 (s, 3H), from 3.22 to 3.45 (partially masked m, 4H), 3.69 (dd, J=6.0 and 8.0 Hz, 1H), 3.99 (dd, J=6.0 and 8.0 Hz, 1H), 4.20 (m, 1H), 4.38 (s, 2H), 4.72 (m, 1H), 7.30 (d, J=9.0 Hz, 4H), 7.36 (d, J=9.0 Hz, 4H), from 7.43 to 7.54 (m, 2H), 7.78 (s, 1H), 7.82 (m, 1H), 8.69 (t, J=6.0 Hz, 1H);
Mass spectrum: ES m/z=618 (MH⁺, base peak);
Optical rotation: α_{D}=+7.2° (c=0.420, DMSO).

### EXAMPLE 4

### (−)-3-({1-[Bis(4-chlorophenyl)methyl]azetidin-3-yl}(methanesulphonyl)amino)-N-(2,3-dihydroxypropyl)benzamide (Compound No. 22)

0.2 g of (+)-3-({1-[bis(4-chlorophenyl)methyl]azetidin-3-yl}(methanesulphonyl)amino)-N-(2,2-dimethyl-1,3-dioxolan-4-ylmethyl)benzamide, 4 cm³ of tetrahydrofuran and 2 cm³ of a 1N solution of hydrochloric acid in ethyl ether are stirred at a temperature in the region of 20° C. for 5 hours. The reaction medium is poured onto an aqueous sodium hydrogencarbonate solution. After separating by settling, the aqueous phase is extracted with ethyl acetate. The organic phases are combined, washed with a saturated aqueous sodium chloride solution, dried over magnesium sulphate, filtered and concentrated to dryness under reduced pressure (5 kPa). 0.15 g of (−)-3-({1-[bis(4-chlorophenyl)methyl]azetidin-3-yl}(methanesulphonyl)amino)-N-(2,3-dihydroxypropyl)benzamide is obtained in the form of a white foam.
¹H NMR spectrum (400 MHz; (δ in ppm); (d₆-DMSO); referenced at 2.50 ppm): 2.70 (t, J=7.5 Hz, 2H), 2.96 (s, 3H), 3.19 (m, 1H), from 3.30 to 3.45 (m, 5H), 3.65 (m, 1H); 4.38 (s, 1H), 4.54 (t, J=6.0 Hz, 1H), 4.72 (m, 1H), 4.79 (d, J=6.0 Hz, 1H), 7.30 (d, J=9.0 Hz, 4H), 7.36 (d, J=9.0 Hz, 4H), from 7.42 to 7.53 (m, 2H), 7.78 (broad s, 1H); 7.83 (broad d, J=8.0 Hz, 1H), 8.47 (t, J=6.0 Hz, 1H);
Mass spectrum: ES m/z=578 (MH⁺, base peak);
Elemental analysis:
Calculated: C: 56.06% H: 5.05% N: 7.26% S: 5.54%
Measured: C: 55.40% H: 5.68% N: 6.87% S: 5.34% H₂O: 1.21%;
Optical rotation: α_{D}=−6.9° (c=0.357, MeOH).

### EXAMPLE 5

### (+)-3-({1-[Bis(4-chlorophenyl)methyl]azetidin-3-yl}(methanesulphonyl)amino)-N-(2,3-dihydroxypropyl)benzamide (Compound No. 23)

0.2 g of (−)-3-({1-[bis(4-chlorophenyl)methyl]azetidin-3-yl}(methanesulphonyl)amino)-N-(2,2-dimethyl-1,3-dioxolan-4-ylmethyl)benzamide, 4 cm³ of tetrahydrofuran and 2 cm³ of a 1N solution of hydrochloric acid in ethyl ether are stirred at a temperature in the region of 20° C. for 5 hours. The reaction medium is poured onto an aqueous sodium hydrogencarbonate solution. After separating by settling, the aqueous phase is extracted with ethyl acetate. The organic phases are combined, washed with a saturated aqueous sodium chloride solution, dried over magnesium sulphate, filtered and concentrated to dryness under reduced pressure (5 kPa). 0.196 g of (+)-3-({1-[bis(4-chlorophenyl)methyl]azetidin-3-yl}(methanesulphonyl)amino)-N-(2,3-dihydroxypropyl)benzamide is obtained in the form of a white foam.
¹H NMR spectrum (400 MHz; (δ in ppm); (d₆-DMSO); referenced at 2.50 ppm): 2.70 (t, J=7.5 Hz, 2H), 2.95 (s, 3H), 3.19 (m, 1H), from 3.30 to 3.45 (m, 5H), 3.63 (m, 1H), 4.38 (s, 1H), 4.54 (t, J=6.0 Hz, 1H), 4.72 (m, 1H), 4.79 (d, J=6.0 Hz, 1H), 7.30 (d, J=9.0 Hz, 4H), 7.35 (d, J=9.0 Hz, 4H), from 7.42 to 7.53 (m, 2H), 7.79 (broad s, 1H), 7.83 (broad d, J=8.0 Hz, 1H), 8.47 (t, J=6.0 Hz, 1H);
Mass spectrum: ES m/z=578 (MH⁺, base peak);
Elemental analysis:
Calculated: C: 56.06% H: 5.05% N: 7.26% S: 5.54%
Measured: C: 54.57% H: 5.11% N: 6.85% S: 4.91%-H₂O: 1.94%;
Optical rotation: α_{D}=+7.0° (c=0.241, MeOH).

### EXAMPLE 6

### 3-({1-[Bis(4-chlorophenyl)methyl]azetidin-3-yl}(methanesulphonyl)amino)-N-(2-hydroxyethyl)benzamide hydrochloride (1:1) (Compound No. 7)

A suspension of 300 mg of 3-[{1-[bis(4-chlorophenyl)methyl]azetidin-3-yl}(methanesulphonyl)amino]benzoic acid and 40 μl of ethanolamine in 5 cm³ of dichloromethane is stirred under an inert atmosphere at a temperature in the region of 20° C. for 10 min 136 mg of 1-(3-dimethylaminopropyl)-3-ethylcarbodiimide hydrochloride in 3 cm³ of dichloromethane are then added. The solution obtained is stirred for 18 hours before again adding 10 μl of ethanolamine After stirring for an additional 24 hours at a temperature in the region of 20° C., the reaction medium is taken up in 40 cm³ of dichloromethane and 10 cm³ of a saturated aqueous sodium chloride solution. After separating by settling, the organic phase is dried over magnesium sulphate, filtered through a sintered glass filter and then concentrated to dryness under reduced pressure to give 0.39 g of a foam. The crude reaction product is purified by flash chromatography on a cartridge comprising 30 g of Merck silica (particle size: 15-40 μm; elution gradient: dichloromethane/methanol 98/2 to 95/5). After concentrating the fractions under reduced pressure, 0.144 g of a colourless oil is obtained, which oil is taken up in 5 cm³ of diethyl ether and 0.65 cm³ of a 2M solution of hydrochloric acid in diethyl ether. After stirring for 10 minutes and then concentrating under vacuum, the residue obtained is again taken up in 0.10 cm³ of a 2M solution of hydrochloric acid in diethyl ether. After application of the above treatment, the new residue obtained is triturated from a pentane/diethyl ether (2/3:1/3) mixture and then dried in an oven under vacuum at a temperature in the region of 40° C. for 2 hours. The hydrochloride salt thus obtained is subjected to the same treatment as above: stirring for 10 minutes in 0.7 cm³ of dichloromethane and 0.1 cm³ of a 2M solution of hydrochloric acid in diethyl ether, concentrating under vacuum, triturating twice from pentane and drying in an oven under vacuum at a temperature in the region of 40° C. for 2 hours 30 minutes. 99 mg of 3-({1-[bis(4-chlorophenyl)methyl]azetidin-3-yl}(methanesulphonyl)amino)-N-(2-hydroxyethyl)benzamide hydrochloride are thus obtained in the form of a yellowish foam.
¹H NMR spectrum (400 MHz; (δ in ppm); (d₆-DMSO); referenced at 2.50 ppm): 3.00 (broad s, 3H), 3.25-3.45 (masked m, 6H), 3.52 (t, J=6,1 Hz, 2H); 4.11 (broad unresolved m, 1H), 4.91 (broad unresolved m, 2H), 7.25-7.64 (m, 10H), 7.76-7.97 (m, 2H); 8.52 (m, 1H);
Mass spectrum: ES m/z=548 (MH⁺, base peak).

### EXAMPLE 7

### (−)-3-({1-[Bis(4-chlorophenyl)methyl]azetidin-3-yl}(methanesulphonyl)amino)-N-(2-hydroxy-1-methylethyl)benzamide (Compound No. 8)

0.227 g of 1-(3-dimethylaminopropyl)-3-ethylcarbodiimide hydrochloride is added to a suspension of 0.5 g of 3-[{1-[bis(4-chlorophenyl)methyl]azetidin-3-yl}(methylsulphonyl)amino]-benzoic acid in 10 cm³ of dichloromethane. A solution of 0.082 g of (2R)-2-aminopropan-1-ol in 1 cm³ of dichloromethane is run onto the reaction medium, which is stirred under an inert atmosphere at a temperature in the region of 20° C. for 96 hours before being concentrated to dryness under reduced pressure (5 kPa). 0.75 g of product is obtained, which product is purified by flash chromatography on a cartridge comprising 30 g of Merck silica (particle size: 15-40 μm; elution gradient: dichloromethane/methanol 98/2 to 95/5). After concentrating the fractions under reduced pressure, 0.205 g of (−)-3-[{1-[bis(4-chlorophenyl)methyl]azetidin-3-yl}(methanesulphonyl)amino]-N-[2-hydroxy-1-methylethyl]benzamide is obtained in the form of a white foam.
¹H NMR spectrum (400 MHz; (δ in ppm); (d₆-DMSO); referenced at 2.50 ppm): 1.13 (d, J=6.5 Hz, 3H), 2.70 (t, J=7.5 Hz, 2H), 2.97 (s, 3H), from 3.22 to 3.40 (partially masked m, 3H), 3.47 (m, 1H), 4.02 (m, 1H), 4.38 (s, 1H), 4.70 (t, J=6.0 Hz, 1H), 4.73 (m, 1H), 7.30 (d, J=9.0 Hz, 4H), 7.35 (d, J=9.0 Hz, 4H), from 7.42 to 7.51 (m, 2H), 7.78 (s, 1H), 7.83 (m, 1H), 8.13 (d, J=8.0 Hz, 1H);
Mass spectrum: ES m/z=562 (MH⁺, base peak);
Elemental analysis:
Calculated: C: 57.65% H: 5.20% N: 7.47% S: 5.70%
Measured: C: 57.56% H: 5.41% N: 7.12% S: 5.50%;
Optical rotation: α_{D}=−3.0° (c=0.371, DMSO).

### EXAMPLE 8

### (+)-3-({1-[Bis(4-chlorophenyl)methyl]azetidin-3-yl}(methanesulphonyl)amino)-N-(2-hydroxy-1-methylethyl)benzamide (Compound No. 9)

1.82 g of 1-(3-dimethylaminopropyl)-3-ethylcarbodiimide hydrochloride are added to a suspension of 4 g of 3-[{1-[bis(4-chlorophenyl)methyl]azetidin-3-yl}(methylsulphonyl)amino]-benzoic acid in 60 cm³ of dichloromethane. A solution of 0.68 cm³ of (2S)-2-aminopropan-1-ol in 3 cm³ of dichloromethane is run dropwise onto the reaction medium, which is stirred under an inert atmosphere at a temperature in the region of 20° C. for 9 hours. The following are then again added: 0.455 g of 1-(3-dimethylaminopropyl)-3-ethylcarbodiimide hydrochloride and 0.184 cm³ of (2S)-2-aminopropan-1-ol. Stirring is continued overnight at then the reaction medium is concentrated to dryness under reduced pressure. 7 g of a white foam are obtained, which product is purified by flash chromatography on a cartridge comprising 400 g of Merck silica (particle size: 15-40 μm; eluent: dichloromethane/methanol 98/2). After concentrating the fractions under reduced pressure, 2.5 g of (+)-3-({1-[bis(4-chlorophenyl)methyl]azetidin-3-yl}(methanesulphonyl)amino)-N-(2-hydroxy-1-methylethyl)benzamide are obtained in the form of a white foam. This batch is combined with two other batches with respective weights of 1.47 g and 0.95 g synthesized according to the same procedure. The 4.92 g of product finally obtained are recrystallized from a water/absolute ethanol mixture to give, after filtration and drying, 4.07 g of (+)-3-({1-[bis(4-chlorophenyl)methyl]azetidin-3-yl}(methanesulphonyl)amino)-N-(2-hydroxy-1-methylethyl)benzamide in the form of a white solid. 2.5 g of the same product, synthesized and recrystallized as above, are added to this batch. The combining of these two batches makes it possible to obtain 6.57 g of a white solid which is recrystallized from pentane. After drying, 6.45 g of (+)-3-({1-[bis(4-chlorophenyl)methyl]azetidin-3-yl}(methanesulphonyl)amino)-N-(2-hydroxy-1-methylethyl)benzamide are finally obtained in the form of a white solid.
M.p.: 192-194° C.;
¹H NMR spectrum (400 MHz; (δ in ppm); (d₆-DMSO); referenced at 2.50 ppm): 1.13 (d, J=6.8 Hz, 3H), 2.70 (t, J=6.8 Hz, 2H), 2.96 (s, 3H), 3.30 (partially masked m, 3H), 3.46 (m, 1H), 4.01 (m, 1H), 4.37 (s, 1H), 4.68-4.77 (m, 2H), 7.30 (d, J=8.6 Hz, 4H), 7.35 (d, J=8.6 Hz, 4H), 7.42-7.52 (m, 2H), 7.77 (broad s, 1H), 7.83 (m, 1H), 8.15 (d, J=7.8 Hz, 1H);
Mass spectrum: ES m/z=562 (MH⁺, base peak);
Elemental analysis:
Calculated: C: 57.65% H: 5.20% N: 7.47% S: 5.70%
Measured: C: 57.66% H: 5.28% N: 7.53% S: 5.70%;
Optical rotation: α_{D}=+5.9° (c=0.401, DMSO).

### EXAMPLE 9

### 3-({1-[Bis(4-chlorophenyl)methyl]azetidin-3-yl}(methanesulphonyl)amino)-N-(2-hydroxy-1-(hydroxymethyl)ethyl)benzamide (Compound No. 17)

67 mg of 1-hydroxybenzotriazole, 0.417 cm³ of triethylamine, 227 mg of 1-(3-dimethylaminopropyl)-3-ethylcarbodiimide hydrochloride and 15 cm³ of tetrahydrofuran are successively added to a suspension of 500 mg of 3-[{1-[bis(4-chlorophenyl)methyl]azetidin-3-yl}(methanesulphonyl)amino]benzoic acid and 135 mg of 2-amino-1,3-propanediol in 10 cm³ of tetrahydrofuran. The reaction mixture is stirred at a temperature in the region of 20° C. for 21 hours. After concentrating the reaction medium to dryness under reduced pressure, the foam obtained is taken up in 100 cm³ of dichloromethane and 30 cm³ of water. After separating by settling, the aqueous phase is extracted twice with 30 cm³ of dichloromethane. The organic phases are combined, washed with 35 cm³ of a saturated aqueous sodium chloride solution, dried over sodium sulphate, filtered and then concentrated to dryness under reduced pressure. 0.69 g of a beige foam is thus obtained, which product is purified by flash chromatography on a cartridge comprising 30 g of Merck silica (eluent: dichloromethane/methanol 94/6). After concentrating the fractions under reduced pressure, the product obtained is recrystallized from an absolute ethanol/water mixture to give, after filtration and drying, 292 mg of 3-({1-[bis(4-chlorophenyl)methyl]azetidin-3-yl}(methanesulphonyl)amino)-N-(2-hydroxy-1-hydroxymethylethyl)benzamide in the form of a white solid.
M.p.: 192-194° C.;
¹H NMR spectrum (400 MHz; (δ in ppm); (d₆-DMSO); referenced at 2.50 ppm): 2.70 (t, J=7.6 Hz, 2H), 2.96 (s, 3H), 3.31-3.37 (m, 2H), 3.41-3.60 (m, 4H), 3.97 (m, 1H), 4.38 (s, 1H), 4.65 (t, J=5.6 Hz, 2H), 4.74 (quin, J=6.8 Hz, 1H), 7.31 (d, J=8.6 Hz, 4H), 7.36 (d, J=8.6 Hz, 4H), 7.43-7.54 (m, 2H), 7.78 (broad s, 1H), 7.85 (m, 1H), 8.04 (d, J=8.3 Hz, 1H);
Mass spectrum: ES m/z=578 (MH⁺, base peak);
Elemental analysis:
Calculated: C: 56.06% H: 5.05% N: 7.26% S: 5.54%
Measured: C: 56.03% H: 5.08% N: 7.28% S: 5.21%.

### EXAMPLE 10

### (+)-3-({1-[Bis(4-chlorophenyl)methyl]azetidin-3-yl}(methanesulphonyl)amino)-5-fluoro-N-(2-hydroxy-1-methylethyl)benzamide (Compound No. 25)

0.137 cm³ of isobutyl chloroformate is added to a solution, cooled to a temperature of between −5° C. and −10° C., of 0.5 g of 3-({1-[bis(4-chlorophenyl)methyl]azetidin-3-yl}(methanesulphonyl)amino)-5-fluorobenzoic acid and 0.173 cm³ of triethylamine in 10 cm³ of tetrahydrofuran. The reaction medium is stirred at a temperature in the region of 0° C. for 1 hour before adding dropwise a solution of 0.112 cm³ of (S)-2-amino-1-propanol in 1 cm³ of tetrahydrofuran. The reaction medium is stirred overnight at a temperature in the region of 20° C. and is then filtered through a sintered glass filter, rinsing being carried out with dichloromethane. The filtrate is concentrated to dryness under vacuum to give 0.7 g of a white foam which is purified by flash chromatography on a cartridge comprising 30 g of Merck silica (particle size: 15-40 μm; eluent: dichloromethane/methanol 98/2). After concentrating the fractions under reduced pressure, a product is obtained which is recrystallized from an absolute ethanol/water mixture. After filtering and drying under vacuum at a temperature in the region of 40° C., 0.240 g of (+)-3-{1-[bis(4-chlorophenyl)methyl]azetidin-3-yl}(methanesulphonyl)amino)-5-fluoro-N-(2-hydroxy-1-methylethyl)benzamide is obtained in the form of a white solid.
M.p: 148-150° C.;
¹H NMR spectrum (400 MHz; (δ in ppm); (d₆-DMSO); referenced at 2.50 ppm): 1.13 (d, J=6.8 Hz, 3H), 2.73 (t, J=7.1 Hz, 2H), 2.99 (s, 3H), 3.28-3.38 (partially masked m, 3H), 3.45 (m, 1H), 4.00 (m, 1H), 4.40 (s, 1H), 4.72 (m, 2H), 7.27-7.34 (d, J=8.6 Hz, 4H), 7.36 (d, J=8.6 Hz, 4H), 7.41 (dt, J=9.6 and 1.8 Hz, 1H), 7.65 (t, J=1.8 Hz, 1H), 7.69 (dd, J=9.6 and 1.8 Hz, 1H), 8.24 (d, J=7.8 Hz, 1H);
Mass spectrum: ES m/z=580 (MH⁺, base peak);
Elemental analysis:
Calculated: C: 55.86% H: 4.86% N: 7.24% S: 5.52%
Measured: C: 55.58% H: 5.13% N: 6.82% S: 5.05%;
Optical rotation: α_{D}=+8.9° (c=0.440, DMSO).

### EXAMPLE 11

### 3-({1-[Bis(4-chlorophenyl)methyl]azetidin-3-yl}(methanesulphonyl)amino)-5-fluoro-N-(2-hydroxy-1-(hydroxymethyl)ethyl)benzamide (Compound No. 26)

52 mg of 1-hydroxybenzotriazole, 0.322 cm³ of triethylamine, 175 mg of 1-(3-dimethylaminopropyl)-3-ethylcarbodiimide hydrochloride and 10 cm³ of tetrahydrofuran are successively added to a suspension of 400 mg of 3-({1-[bis(4-chlorophenyl)methyl]azetidin-3-yl}(methanesulphonyl)amino)-5-fluorobenzoic acid and 104 mg of 2-amino-1,3-propanediol in 10 cm³ of tetrahydrofuran. The reaction mixture is stirred at a temperature in the region of 20° C. for 24 h. After filtering the reaction medium and then concentrating to dryness under reduced pressure, 0.80 g of a yellowish oil is obtained, which oil is purified by flash chromatography on a cartridge comprising 70 g of Merck silica (eluent: dichloromethane/methanol 98/2). After concentrating the fractions under reduced pressure, the product obtained is taken up in diethyl ether and then dichloromethane. After concentrating to dryness under reduced pressure, a foam is obtained, which foam is recrystallized from an absolute ethanol/water mixture to give, after filtering and drying, 255 mg of 3-({1-[bis(4-chlorophenyl)methyl]azetidin-3-yl}(methanesulphonyl)amino)-5-fluoro-N-(2-hydroxy-1-(hydroxymethyl)ethyl)benzamide in the form of a white foam.
M.p.: 144-146° C.;
¹H NMR spectrum (400 MHz; (δ in ppm); (d₆-DMSO); referenced at 2.50 ppm): 2.73 (t, J=7.3 Hz, 2H), 3.00 (s, 3H), 3.35 (t, J=7.3 Hz, 2H), 3.46-3.59 (m, 4H), 3.96 (m, 1H), 4.40 (s, 1H), 4.65 (t, J=5.6 Hz, 2H), 4.73 (m, 1H), 7.31 (d, J=8.3 Hz, 4H), 7.37 (d, J=8.3 Hz, 4H), 7.41 (dt, J=9.2 and 1.8 Hz, 1H), 7.66 (t, J=1.8 Hz, 1H), 7.71 (dt, J=8.6 and 1.8 Hz, 1H), 8.14 (d, J=7.8 Hz, 1H);
Mass spectrum: ES m/z=596 (MH⁺, base peak);
Elemental analysis:
Calculated: C: 54.37% H: 4.73% N: 7.04% S: 5.38%
Measured: C: 52.60% H: 4.95% N: 6.84% S: 5.06% H₂O: 3.36%.

### EXAMPLE 12

### 3-({1-[Bis(4-chlorophenyl)methyl]azetidin-3-yl}(methanesulphonyl)amino)-5-fluoro-N-(2-hydroxy-1-hydroxymethyl-1-methylethyl)benzamide (Compound No. 27)

0.143 cm³ of isobutyl chloroformate is added to a stirred solution, cooled to a temperature of between −10° C. and −20° C., of 0.5 g of 3-({1-[bis(4-chlorophenyl)methyl]azetidin-3-yl}(methanesulphonyl)amino)-5-fluorobenzoic acid and 0.226 cm³ of triethylamine in 5 cm³ of tetrahydrofuran. The reaction medium is stirred at a temperature in the region of 0° C. for 1 hour before adding dropwise, at a temperature of between −10° C. and −20° C., a solution of 0.15 g of 2-amino-2-methyl-1,3-propanediol in 2 cm³ of tetrahydrofuran. The reaction medium is stirred at a temperature in the region of 20° C. for 20 hours and is then filtered through a sintered glass filter, rinsing being carried out with dichloromethane. The filtrate is concentrated to dryness under vacuum to give 0.73 g of a white foam, which is purified by flash chromatography on a cartridge comprising 70 g of Merck silica (particle size: 15-40 μm; eluent: ethyl acetate/methanol 98/2). After concentrating the fractions under reduced pressure and then drying under vacuum at a temperature in the region of 40° C., 0.404 g of 3-({1-[bis(4-chlorophenyl)methyl]azetidin-3-yl}(methanesulphonyl)amino)-5-fluoro-N-(2-hydroxy-1-hydroxymethyl-1-methylethyl)benzamide is obtained in the form of a white foam.
M.p.: 159-161° C.;
¹H NMR spectrum (400 MHz; (δ in ppm); (d₆-DMSO); referenced at 2.50 ppm): 1.26 (s, 3H), 2.73 (t, J=7.3 Hz, 2H), 2.99 (s, 3H), 3.29-3.41 (partially masked m, 2H), 3.56 (dd, J=10.8 and 5.4 Hz, 2H); 3.61 (dd, J=10.8 and 5.4 Hz, 2H), 4.41 (s, 1H); 4.74 (m, 3H), 7.31 (d, J=8.6 Hz, 4H), 7.37 (d, J=8.6 Hz, 4H), 7.40 (dd, J=9.6 and 1.8 Hz, 1H), 7.49 (s, 1H), 7.58 (t, J=1.8 Hz, 1H), 7.64 (dt, J=9.0 and 1.8 Hz, 1H);
Mass spectrum: ES m/z=610 (MH⁺, base peak).

The chemical structures (I) and the physical properties (Table 1A) of a few examples of compounds according to the invention are illustrated in the following Table 1. In this table:

In the “Salt” column of Table 1, B represents the product obtained in the base form.
R represents a methyl group;
R3 and R4 each represent a phenyl group substituted by a chlorine atom in the para position.

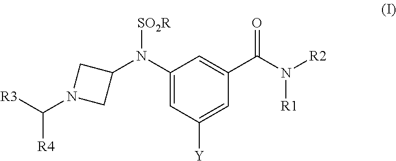

TABLE 1

| No. | 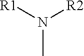 | Y | Chirality | Salt |
|---|---|---|---|---|
| 1 | 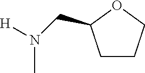 | H | Chiral (+) | B |
| 2 | 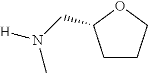 | H | Chiral (−) | B |
| 3 | 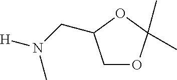 | H | — | B |
| 4 | 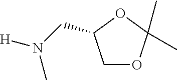 | H | Chiral (+) | B |
| 5 | 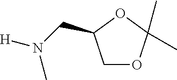 | H | Chrial (−) | B |
| 6 | 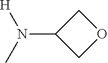 | H | — | B |
| 7 | —NHCH₂CH₂OH | H | — | HCl (1:1) |
| 8 | 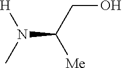 | H | Chiral (−) | B |
| 9 | 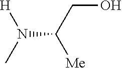 | H | Chiral (+) | B |
| 10 | 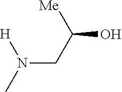 | H | Chiral (−) | B |
| 11 | 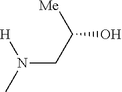 | H | Chiral (+) | B |
| 12 | —NHCH₂—CH(OH)—CF₃ | H | — | B |
| 13 | —NHCH₂C(Me)₂OH | H | — | B |
| 14 | 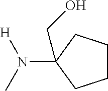 | H | — | B |
| 15 | 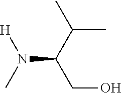 | H | Chiral (S) | B |
| 16 | —NHC(Me)₂CH₂OH | H | — | B |
| 17 | —NHCH(CH₂OH)₂ | H | — | B |
| 18 | —NHC(Me)(CH₂OH)₂ | H | — | B |
| 19 | —NHC(CH₂OH)₃ | H | — | B |
| 20 | 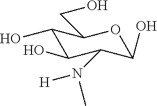 | H | Chiral | B |
| 21 | 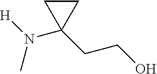 | H | — | B |
| 22 | 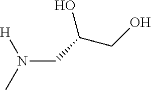 | H | Chiral (−) | B |
| 23 | 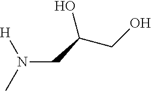 | H | Chiral (+) | B |
| 24 | —NHCH₂CH₂OMe | H | — | B |
| 25 | 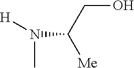 | F | Chiral (+) | B |
| 26 | —NHCH(CH₂OH)₂ | F | — | B |
| 27 | —NHCMe(CH₂OH)₂ | F | — | B |
| 28 | 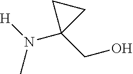 | H | — | B |
| 29 | 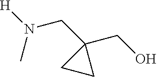 | H | — | B |
| 30 | NHCH₂—CH(OH)—CF₃ | F | — | B |
| 31 | 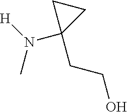 | F | — | B |
| 32 | 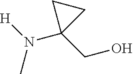 | F | — | B |
| 33 | 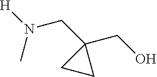 | F | — | B |
| 34 | 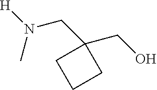 | F | — | B |
| 35 | —NHCH₂CH₂OH | CF₃ | — | B |
| 36 | 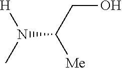 | CF₃ | Chiral (S) | B |
| 37 | —NHCH(CH₂OH)₂ | CF₃ | — | B |
| 38 | —NHCMe(CH₂OH)₂ | CF₃ | — | B |
| 39 | 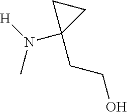 | CF₃ | — | B |
| 40 | 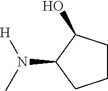 | CF₃ | (+/−)-cis | B |
| 41 | 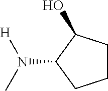 | CF₃ | (+/−)-trans | B |
| 42 | 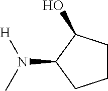 | F | (+/−)-cis | B |
| 43 | 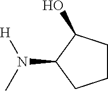 | F | Chiral (−) | B |
| 44 | 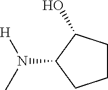 | F | Chiral (+) | B |
| 45 | 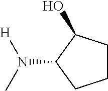 | F | (+/−)-trans | B |
| 46 | 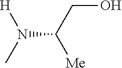 | OCH₃ | Chiral (S) | B |
| 47 | 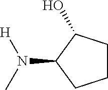 | F | (+/−)-trans | B |
| 48 | 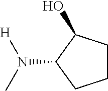 | F | Chiral (−) | B |
| 49 | 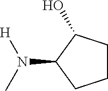 | F | Chiral (+) | B |

TABLE 1A

| No. | Characterizations |
|---|---|
| 1 | ¹H NMR spectrum (300 MHz; (δ in ppm); (d₆-DMSO); referenced at |
| | 2.50 ppm): 1.59 (m, 1H), from 1.72 to 1.98 (m, 3H), 2.71 (t, J = 7.5 Hz, |
| | 2H), 2.97 (s, 3H), from 3.23 to 3.50 (partially masked m, 2H), 3.62 (m, |
| | 1H), 3.79 (m, 1H), 3.99 (m, 1H), 4.35 (s, 1H), 4.73 (m, 1H), 7.30 (d, J = 9.0 Hz, |
| | 4H), 7.35 (d, J = 9.0 Hz, 4H), from 7.42 to 7.53 (m, 2H), |
| | 7.79 (s, 1H), 7.83 (m, 1H), 8.60 (t, J = 6.0 Hz, 1H), Mass spectrum: ES |
| | m/z = 588 (MH⁺, base peak); Elemental analysis: Calculated: C: 59.18% |
| | H: 5.31% N: 7.14% S: 5.45%, Measured: C: 58.82% H: 5.54% N: |
| | 7.10% S: 5.21%, Optical rotation: α_{D} = + 13.5° (c = 1.017, MeOH) |
| 2 | 1H NMR spectrum (300 MHz, (δ in ppm), (d₆-DMSO), referenced at |
| | 2.50 ppm): 1.58 (m, 1H), from 1.72 to 1.98 (m, 3H), 2.71 (t, J = 7.5 Hz, |
| | 2H), 2.96 (s, 3H), from 3.23 to 3.45 (partially masked m, 2H), 3.61 (m, |
| | 1H), 3.77 (m, 1H), 3.98 (m, 1H), 4.37 (s, 1H), 4.73 (m, 1H), 7.30 (d, J = 9.0 Hz, |
| | 4H), 7.35 (d, J = 9.0 Hz, 4H), from 7.42 to 7.53 (m, 2H), |
| | 7.79 (s, 1H), 7.84 (m, 1H), 8.60 (t, J = 6.0 Hz, 1H), Mass spectrum: ES |
| | m/z = 588 (MH⁺, base peak); Optical rotation: α_{D} = − 12.4° (c = 0.983, |
| | MeOH) |
| 3 | 1H NMR spectrum (400 MHz; (δ in ppm); (d₆-DMSO); referenced at |
| | 2.50 ppm): 1.26 (s, 3H), 1.32 (s, 3H), 2.69 (m, 2H), 2.96 (s, 3H), from |
| | 3.23 to 3.48 (partially masked m, 4H), 3.68 (dd, J = 6.0 and 8.5 Hz, |
| | 1H), 3.99 (dd, J = 6.0 and 8.5 Hz, 1H), 4.21 (m, 1H), 4.38 (s, 1H), |
| | 4.72 (m, 1H), 7.31 (d, J = 9.0 Hz, 4H), 7.37 (d, J = 9.0 Hz, 4H), from 7.45 to |
| | 7.54 (m, 2H), 7.78 (s, 1H), 7.83 (m, 1H), 8.67 (t, J = 6.0 Hz, 1H), Mass |
| | spectrum: ES m/z = 618 (MH⁺, base peak); Elemental analysis: |
| | Calculated: C: 58.25% H: 5.38% N: 6.79%; Measured: C: 58.03% H: |
| | 5.27% N: 6.73% |
| 4 | ¹H NMR spectrum (400 MHz; (δ in ppm); (d₆-DMSO); referenced at |
| | 2.50 ppm): 1.26 (s, 3H), 1.31 (s, 3H), 2.69 (m, 2H), 2.96 (s, 3H), from |
| | 3.22 to 3.45 (partially masked m, 4H), 3.69 (dd, J = 6.0 and 8.0 Hz, |
| | 1H), 3.99 (dd, J = 6.0 and 8.0 Hz, 1H), 4.20 (m, 1H); 4.38 (s, 2H), |
| | 4.72 (m, 1H), 7.30 (d, J = 9.0 Hz, 4H), 7.36 (d, J = 9.0 Hz, 4H), from 7.43 to |
| | 7.54 (m, 2H), 7.78 (s, 1H), 7.82 (m, 1H), 8.69 (t, J = 6.0 Hz, 1H), Mass |
| | spectrum: ES m/z = 618 (MH⁺, base peak); Optical rotation: α_{D} = + 7.2° (c = 0.420, |
| | DMSO) |
| 5 | ¹H NMR spectrum (400 MHz; (δ in ppm); (d₆-DMSO); referenced at |
| | 2.50 ppm): 1.26 (s, 3H), 1.31 (s, 3H), 2.69 (m, 2H), 2.97 (s, 3H), from |
| | 3.20 to 3.48 (partially masked m, 4H), 3.69 (dd, J = 6.0 and 8.0 Hz, |
| | 1H), 3.99 (dd, J = 6.0 and 8.0 Hz, 1H), 4.20 (m, 1H), 4.38 (s, 2H), |
| | 4.72 (m, 1H), 7.30 (d, J = 9.0 Hz, 4H), 7.35 (d, J = 9.0 Hz, 4H), from 7.43 to |
| | 7.54 (m, 2H), 7.78 (s, 1H), 7.83 (m, 1H), 8.69 (t, J = 6.0 Hz, 1H), Mass |
| | spectrum: ES m/z = 618 (MH⁺, base peak); Optical rotation: α_{D} = − 4.5° (c = 0.438, |
| | DMSO) |
| 6 | M.p.: 105-107° C.; ¹H NMR spectrum (400 MHz; (δ in ppm); (d₆- |
| | DMSO); referenced at 2.50 ppm): 2.70 (t, J = 7.6 Hz, 2H), 2.96 (s, 3H), |
| | 3.34 (t, J = 7.6 Hz, 2H), 4.37 (s, 1H), 4.60 (t, J = 6.8 Hz, 2H), 4.74 (m, |
| | 1H), 4.78 (t, J = 6.8 Hz, 2H), 5.00 (m, 1H), 7.31 (d, J = 8.6 Hz, 4H), |
| | 7.35 (d, J = 8.6 Hz, 4H), from 7.47 to 7.55 (m, 2H), 7.79 (m, 1H), |
| | 7.87 (m, 1H), 9.13 (d, J = 6.4 Hz, 1H), Mass spectrum: ES m/z = 560 (MH⁺, |
| | base peak) |
| 7 | ¹H NMR spectrum (400 MHz; (δ in ppm); (d₆-DMSO); referenced at |
| | 2.50 ppm): 3.00 (broad s, 3H), from 3.25 to 3.45 (masked m, 6H), |
| | 3.52 (t, J = 6.1 Hz, 2H), 4.11 (broad unresolved m, 1H), 4.91 (broad |
| | unresolved m, 2H), from 7.25 to 7.64 (m, 10H), from 7.76 to 7.97 (m, |
| | 2H), 8.52 (m, 1H), Mass spectrum: ES m/z = 548 (MH⁺, base peak) |
| 8 | ¹H NMR spectrum (400 MHz; (δ in ppm); (d₆-DMSO); referenced at |
| | 2.50 ppm): 1.13 (d, J = 6.5 Hz, 3H), 2.70 (t, J = 7.5 Hz, 2H), 2.97 (s, |
| | 3H), from 3.22 to 3.40 (partially masked m, 3H), 3.47 (m, 1H), |
| | 4.02 (m, 1H), 4.38 (s, 1H), 4.70 (t, J = 6.0 Hz, 1H), 4.73 (m, 1H), 7.30 (d, J = 9.0 Hz, |
| | 4H), 7.35 (d, J = 9.0 Hz, 4H), from 7.42 to 7.51 (m, 2H), |
| | 7.78 (s, 1H), 7.83 (m, 1H), 8.13 (d, J = 8.0 Hz, 1H), Mass spectrum: ES |
| | m/z = 562 (MH⁺, base peak), Elemental analysis: Calculated: C: 57.65% |
| | H: 5.20% N: 7.47% S: 5.70%; Measured: C: 57.56% H: 5.41% N: |
| | 7.12% S: 5.50%; Optical rotation: α_{D} = − 3.0° (c = 0.371, DMSO) |
| 9 | M.p.: 192-194° C.; ¹H NMR spectrum (400 MHz; (δ in ppm); (d₆- |
| | DMSO); referenced at 2.50 ppm): 1.13 (d, J = 6.8 Hz, 3H), 2.70 (t, J = 6.8 Hz, |
| | 2H), 2.96 (s, 3H), 3.30 (partially masked m, 3H), 3.46 (m, 1H), |
| | 4.01 (m, 1H), 4.37 (s, 1H), from 4.68 to 4.77 (m, 2H), 7.30 (d, J = 8.6 Hz, |
| | 4H), 7.35 (d, J = 8.6 Hz, 4H), from 7.42 to 7.52 (m, 2H), |
| | 7.77 (broad s, 1H), 7.83 (m, 1H), 8.15 (d, J = 7.8 Hz, 1H), Mass spectrum: |
| | ES m/z = 562 (MH⁺, base peak); Elemental analysis: Calculated: C: |
| | 57.65% H: 5.20% N: 7.47% S: 5.70%; Measured: C: 57.66% H: 5.28% |
| | N: 7.53% S: 5.70%; Optical rotation: α_{D} = + 5.9° (c = 0.401, DMSO) |
| 10 | ¹H NMR spectrum (400 MHz; (δ in ppm); (d₆-DMSO); referenced at |
| | 2.50 ppm): 1.03 (d, J = 6.3 Hz, 3H), 2.67 (m, 2H), 2.93 (s, 3H); |
| | 3.17 (m, 1H), from 3.36 to 3.22 (m, 2H), 3.76 (m, 1H), 4.34 (s, 1H), from |
| | 4.77 to 4.64 (m, 2H), from 7.37 to 7.22 (m, 8H), from 7.48 to 7.40 (m, |
| | 2H), from 7.85 to 7.71 (m, 2H), 8.43 (t, J = 5.8 Hz, 1H); Mass |
| | spectrum: ES m/z = 562 (MH⁺, base peak); Optical rotation: α_{D} = − 8.0° (c = 0.83, |
| | MeOH) |
| 11 | ¹H NMR spectrum (400 MHz; (δ in ppm); (d₆-DMSO); referenced at |
| | 2.50 ppm): 1.03 (d, J = 6.2 Hz, 3H), 2.67 (t, J = 7.4 Hz, 2H), 2.93 (s, |
| | 3H), 3.17 (m, 1H), from 3.37 to 3.22 (m, 2H), 3.76 (m, 1H), 4.34 (s, |
| | 1H), 4.70 (m, 2H), from 7.52 to 7.20 (m, 10H), from 7.84 to 7.72 (m, |
| | 2H), 8.44 (t, J = 5.7 Hz, 1H), Mass spectrum: ES m/z = 562 (MH⁺, base |
| | peak); Optical rotation: α_{D} = + 7.4° (c = 0.948, MeOH) |
| 12 | ¹H NMR spectrum (400 MHz; (δ in ppm); (d₆-DMSO); referenced at |
| | 2.50 ppm): 2.67 (m, 2H), 2.93 (s, 3H), from 3.37 to 3.19 (m, 3H), |
| | 3.59 (m, 1H), 4.16 (m, 1H), 4.34 (s, 1H), 4.70 (m, 1H), 6.46 (d, J = 6 Hz, |
| | 1H), from 7.38 to 7.22 (m, 8H), from 7.51 to 7.42 (m, 2H), from 7.86 |
| | to 7.72 (m, 2H), 8.75 (t, J = 5.6 Hz, 1H), Mass spectrum: ES m/z = 616 (MH⁺, |
| | base peak) |
| 13 | M.p.: 140° C.; ¹H NMR spectrum (400 MHz; (δ in ppm); (d₆-DMSO); |
| | referenced at 2.50 ppm): 1.10 (s, 6H), 2.71 (t large, J = 7.6 Hz, 2H), |
| | 2.96 (s, 3H), 3.25 (d, J = 6.4 Hz, 2H), from 3.28 to 3.36 (partially |
| | masked m, 2H), 4.38 (s, 1H), 4.55 (s, 1H), 4.74 (m 1H), 7.30 (d, J = 8.3 Hz, |
| | 4H), 7.35 (d, J = 8.3 Hz, 4H), from 7.44 to 7.52 (m, 2H), |
| | 7.79 (broad s, 1H), 7.85 (dt, J = 7.5 and 1.9 Hz, 1H), 8.33 (t, J = 6.4 Hz, |
| | 1H), Mass spectrum: ES m/z = 576 (MH⁺, base peak); Elemental |
| | analysis: Calculated: C: 58.33% H: 5.42% N: 7.29% S: 5.56%; |
| | Measured: C: 58.66% H: 5.53% N: 7.36% S: 5.40% |
| 14 | M.p.: 189° C.; ¹H NMR spectrum (400 MHz; (δ in ppm); (d₆-DMSO); |
| | referenced at 2.50 ppm): from 1.47 to 1.80 (m, 6H), 1.99 (m, 2H), |
| | 2.70 (t, J = 7.5 Hz, 2H), 2.96 (s, 3H), 3.26 to 3.36 (partially masked m, 2H), |
| | 3.58 (d, J = 5.9 Hz, 2H), 4.38 (s, 1H), 4.74 (m, 1H), 4.82 (t, J = 5.9 Hz, |
| | 1H), 7.30 (d, J = 8.6 Hz, 4H), 7.36 (d, J = 8.6 Hz, 4H), from 7.42 to |
| | 7.54 (m, 2H), 7.72 (broad s, 1H), from 7.77 to 7.82 (m, 2H), Mass |
| | spectrum: ES m/z = 602 (MH⁺, base peak) |
| 15 | M.p.: 164° C.; ¹H NMR spectrum (400 MHz; (δ in ppm); (d₆-DMSO); |
| | referenced at 2.50 ppm): 0.87 (d, J = 6.8 Hz, 3H), 0.91 (d, J = 6.8 Hz, |
| | 3H), 1.92 (m, 1H), 2.71 (m, 2H), 2.96 (s, 3H), from 3.26 to |
| | 3.36 (partially masked m, 2H), 3.52 (m, 2H), 3.80 (m, 1H), 4.38 (s, 1H), |
| | 4.57 (broad t, J = 5.6 Hz, 1H), 4.74 (m, 1H), 7.30 (d, J = 8.6 Hz, 4H), |
| | 7.35 (d, J = 8.6 Hz, 4H), from 7.43 to 7.52 (m, 2H), 7.78 (broad s, 1H), |
| | 7.85 (m, 1H), 8.03 (d, J = 8.8 Hz, 1H), Mass spectrum: ES m/z = 590 (MH⁺, |
| | base peak); Elemental analysis: Calculated: C: 58.98% H: |
| | 5.63% N: 7.12% S: 5.43%; Measured: C: 58.94% H: 6.06% N: 7.12% |
| | S: 5.21% H₂O: 1.04%; Optical rotation: α_{D} = 0° (c = 0.405, DMSO) |
| 16 | M.p.: 162° C.; ¹H NMR spectrum (400 MHz; (δ in ppm); (d₆-DMSO); |
| | referenced at 2.50 ppm): 1.32 (s, 6H), 2.72 (t, J = 6.8 Hz, 2H), 2.97 (s, |
| | 3H), from 3.29 to 3.39 (partially masked m, 2H), 3.53 (d, J = 5.5 Hz, |
| | 2H), 4.40 (s, 1H), 4.76 (m 1H), 4.90 (t, J = 5.5 Hz, 1H), 7.33 (d, J = 8.6 Hz, |
| | 4H), 7.37 (d, J = 8.6 Hz, 4H), from 7.43 to 7.50 (m, 2H), 7.60 (s, |
| | 1H), 7.72 (broad s, 1H), 7.79 (m, 1H), Mass spectrum: ES m/z = 576 (MH⁺, |
| | base peak); Elemental analysis: Calculated: C: 58.33% H: |
| | 5.42% N: 7.29% S: 5.56%; Measured: C: 58.51% H: 5.63% N: 7.22% |
| | S: 5.34% |
| 17 | M.p.: 192-194° C.; ¹H NMR spectrum (400 MHz; (δ in ppm); (d₆- |
| | DMSO); referenced at 2.50 ppm): 2.70 (t, J = 7.6 Hz, 2H), 2.96 (s, 3H), |
| | from 3.31 to 3.37 (m, 2H), from 3.41 to 3.60 (m, 4H), 3.97 (m, 1H), |
| | 4.38 (s, 1H), 4.65 (t, J = 5.6 Hz, 2H), 4.74 (quin, J = 6.8 Hz, 1H), |
| | 7.31 (d, J = 8.6 Hz, 4H), 7.36 (d, J = 8.6 Hz, 4H), from 7.43 to 7.54 (m, 2H), |
| | 7.78 (broad s, 1H), 7.85 (m, 1H), 8.04 (d, J = 8.3 Hz, 1H), Mass |
| | spectrum: ES m/z = 578 (MH⁺, base peak); Elemental analysis: |
| | Calculated: C: 56.06% H: 5.05% N: 7.26% S: 5.54%; Measured: C: |
| | 56.03% H: 5.08% N: 7.28% S: 5.21% |
| 18 | M.p.: 192° C.; ¹H NMR spectrum (300 MHz; (δ in ppm); (d₆-DMSO); |
| | referenced at 2.50 ppm): 1.27 (s, 3H), 2.71 (t, J = 7.6 Hz, 2H), 2.96 (s, |
| | 3H), from 3.28 to 3.35 (partially masked m, 2H), from 3.52 to 3.68 (m, |
| | 4H), 4.39 (s, 1H), from 4.64 to 4.89 (m, 3H), 7.31 (d, J = 8.7 Hz, 4H), |
| | 7.36 (d, J = 8.7 Hz, 4H), 7.42 (s, 1H), from 7.43 to 7.51 (m, 2H), |
| | 7.70 (broad s, 1H), 7.77 (m, 1H); Mass spectrum: ES m/z = 592 (MH⁺, base |
| | peak); Elemental analysis: Calculated: C: 56.76% H: 5.27% N: 7.09% |
| | S: 5.41%; Measured: C: 56.80%-H: 5.39% N: 6.99% S: 5.13% |
| 19 | ¹H NMR spectrum (400 MHz; (δ in ppm); (d₆-DMSO); referenced at |
| | 2.50 ppm): 2.70 (t, J = 7.3 Hz, 2H), 2.96 (s, 3H), from 3.28 to |
| | 3.35 (partially masked m, 2H), 3.69 (d, J = 5.8 Hz, 6H), 4.39 (s, 1H), |
| | 4.72 (t, J = 5.8 Hz, 3H), 4.75 (m, 1H), 7.30 (d, J = 8.8 Hz, 4H), |
| | 7.31 (masked s, 1H), 7.36 (d, J = 8.8 Hz, 4H), from 7.44 to 7.52 (m, 2H), |
| | 7.70 (s, 1H), 7.76 (m, 1H), Mass spectrum: ES m/z = 608 (MH⁺, base |
| | peak) |
| 20 | ¹H NMR spectrum (400 MHz; (δ in ppm); (d₆-DMSO); referenced at |
| | 2.50 ppm): Mixture 70% α anomer 30% β anomer with: 2.70 (m, 2H), |
| | 2.96 (s, 3H), from 3.09 to 3.87 (partially masked m, 8H); 4.38 (s, 1H); |
| | from 4.40 to 5.10 (m, 5H), 6.46 (broad d, J = 3.7 Hz, 0.7H), 6.55 (d, J = 6.8 Hz, |
| | 0.3H), 7.31 (d, J = 8.6 Hz, 4H), 7.36 (d, J = 8.6 Hz, 4H), |
| | from 7.42 to 7.52 (m, 2H), from 7.74 to 7.92 (m, 2H), 8.16 (d, J = 7.8 Hz, |
| | 0.7H), 8.25 (d, J = 8.8 Hz, 0.3H), Mass spectrum: ES m/z = 666 (MH⁺, |
| | base peak) |
| 21 | ¹H NMR spectrum (400 MHz; (δ in ppm); (d₆-DMSO); referenced at |
| | 2.50 ppm): 0.72 (m, 4H), 1.76 (t, J = 6.9 Hz, 2H), 2.67 (t, J = 7.4 Hz, |
| | 2H), 2.97 (s, 3H), 3.34 (m, 2H), 3.53 (quartet, J = 6.7 Hz, 2H), 4.35 (t, |
| | J = 5.4 Hz, 1H), 4.38 (s, 1H), 4.73 (quintuplet, J = 6.6 Hz, 1H), from |
| | 7.38 to 7.22 (m, 8H), 7.47 (m, 2H), 7.74 (m, 1H), 7.80 (m, 1H), 8.68 (s, |
| | 1H), Mass spectrum: ES m/z = 588 (MH⁺, base peak) |
| 22 | ¹H NMR spectrum (400 MHz; (δ in ppm); (d₆-DMSO); referenced at |
| | 2.50 ppm): 2.70 (t, J = 7.5 Hz, 2H), 2.96 (s, 3H), 3.19 (m, 1H), from |
| | 3.30 to 3.45 (m, 5H), 3.65 (m, 1H), 4.38 (s, 1H), 4.54 (t, J = 6.0 Hz, |
| | 1H), 4.72 (m, 1H), 4.79 (d, J = 6.0 Hz, 1H), 7.30 (d, J = 9.0 Hz, 4H), |
| | 7.36 (d, J = 9.0 Hz, 4H), from 7.42 to 7.53 (m, 2H), 7.78 (broad s, 1H), |
| | 7.83 (broad d, J = 8.0 Hz, 1H), 8.47 (t, J = 6.0 Hz, 1H), Mass spectrum: |
| | ES m/z = 578 (MH⁺, base peak); Elemental analysis: Calculated: C: |
| | 56.06% H: 5.05% N: 7.26% S: 5.54%; Measured: C: 55.40% H: 5.68% |
| | N: 6.87% S: 5.34% H₂O: 1.21%; Optical rotation: α_{D} = − 6.9° (c = 0.357, |
| | MeOH) |
| 23 | ¹H NMR spectrum (400 MHz; (δ in ppm); (d₆-DMSO); referenced at |
| | 2.50 ppm): 2.70 (t, J = 7.5 Hz, 2H), 2.95 (s, 3H), 3.19 (m, 1H), from |
| | 3.30 to 3.45 (m, 5H), 3.63 (m, 1H), 4.38 (s, 1H), 4.54 (t, J = 6.0 Hz, |
| | 1H), 4.72 (m, 1H), 4.79 (d, J = 6.0 Hz, 1H), 7.30 (d, J = 9.0 Hz, 4H), |
| | 7.35 (d, J = 9.0 Hz, 4H), from 7.42 to 7.53 (m, 2H), 7.79 (broad s, 1H), |
| | 7.83 (broad d, J = 8.0 Hz, 1H), 8.47 (t, J = 6.0 Hz, 1H), Mass spectrum: |
| | ES m/z = 578 (MH⁺, base peak); Elemental analysis: Calculated: C: |
| | 56.06% H: 5.05% N: 7.26% S: 5.54%; Measured: C: 54.57% H: 5.11% |
| | N: 6.85% S: 4.91% H₂O: 1.94%; Optical rotation: α_{D} = + 7° (c = 0.241, |
| | MeOH) |
| 24 | ¹H NMR spectrum (300 MHz; (δ in ppm); (d₆-DMSO); referenced at |
| | 2.50 ppm): 2.71 (t, J = 7.5 Hz, 2H), 2.95 (s, 3H), 3.25 (s, 3H), |
| | 3.32 (partially masked t, J = 7.5 Hz, 2H), 3.43 (m, 4H), 4.38 (s, 1H), |
| | 4.72 (m, 1H), 7.30 (d, J = 9.0 Hz, 4H), 7.35 (d, J = 9.0 Hz, 4H), from 7.42 to |
| | 7.53 (m, 2H), 7.78 (s, 1H), 7.82 (m, 1H), 8.58 (t, J = 6.0 Hz, 1H), Mass |
| | spectrum: ES m/z = 562 (MH⁺, base peak); Elemental analysis: |
| | Calculated: C: 57.65% H: 5.20% N: 7.47% S: 5.70% Cl: 12.61%; |
| | Measured: C: 57.44% H: 5.36% N: 7.36% S: 5.29% Cl: 12.49% |
| 25 | M.p.: 148-150° C.; ¹H NMR spectrum (400 MHz; (δ in ppm); (d₆- |
| | DMSO); referenced at 2.50 ppm): 1.13 (d, J = 6.8 Hz, 3H), 2.73 (t, J = 7.1 Hz, |
| | 2H), 2.99 (s, 3H), from 3.28 to 3.38 (partially masked m, 3H), |
| | 3.45 (m, 1H), 4.00 (m, 1H), 4.40 (s, 1H), 4.72 (m, 2H), from 7.27 to |
| | 7.34 (d, J = 8.6 Hz, 4H), 7.36 (d, J = 8.6 Hz, 4H), 7.41 (dt, J = 9.6 and |
| | 1.8 Hz, 1H), 7.65 (t, J = 1.8 Hz, 1H), 7.69 (dd, J = 9.6 and 1.8 Hz, 1H), |
| | 8.24 (d, J = 7.8 Hz, 1H), Mass spectrum: ES m/z = 580 (MH⁺, base |
| | peak); Elemental analysis: Calculated: C: 55.86% H: 4.86% N: 7.24% |
| | S: 5.52%; Measured: C: 55.58% H: 5.13% N: 6.82% S: 5.05%; Optical |
| | rotation: α_{D} = + 8.9° (c = 0.440, DMSO) |
| 26 | M.p.: 144-146° C.; ¹H NMR spectrum (400 MHz; (δ in ppm); (d₆- |
| | DMSO); referenced at 2.50 ppm): 2.73 (t, J = 7.3 Hz, 2H), 3.00 (s, 3H), |
| | 3.35 (t, J = 7.3 Hz, 2H), from 3.46 to 3.59 (m, 4H), 3.96 (m, 1H), |
| | 4.40 (s, 1H), 4.65 (t, J = 5.6 Hz, 2H), 4.73 (m, 1H), 7.31 (d, J = 8.3 Hz, 4H), |
| | 7.37 (d, J = 8.3 Hz, 4H), 7.41 (dt, J = 9.2 and 1.8 Hz, 1H), 7.66 (t, J = 1.8 Hz, |
| | 1H), 7.71 (dt, J = 8.6 and 1.8 Hz, 1H), 8.14 (d, J = 7.8 Hz, 1H), |
| | Mass spectrum: ES m/z = 596 (MH⁺, base peak); Elemental analysis: |
| | Calculated: C: 54.37% H: 4.73% N: 7.04% S: 5.38%; Measured: C: |
| | 52.60% H: 4.95% N: 6.84% S: 5.06% H₂O: 3.36% |
| 27 | M.p.: 159-161° C.; ¹H NMR spectrum (400 MHz; (δ in ppm); (d₆- |
| | DMSO); referenced at 2.50 ppm): 1.26 (s, 3H); 2.73 (t, J = 7.3 Hz, 2H), |
| | 2.99 (s, 3H), from 3.29 to 3.41 (partially masked m, 2H), 3.56 (dd, J = 10.8 |
| | and 5.4 Hz, 2H), 3.61 (dd, J = 10.8 and 5.4 Hz, 2H), 4.41 (s, 1H), |
| | 4.74 (m, 3H), 7.31 (d, J = 8.6 Hz, 4H), 7.37 (d, J = 8.6 Hz, 4H), |
| | 7.40 (dd, J = 9.6 and 1.8 Hz, 1H), 7.49 (s, 1H), 7.58 (t, J = 1.8 Hz, 1H), |
| | 7.64 (dt, J = 9.0 and 1.8 Hz, 1H), Mass spectrum: ES m/z = 610 (MH⁺, base |
| | peak) |
| 28 | ¹H NMR spectrum (400 MHz; (δ in ppm); (d₆-DMSO); referenced at |
| | 2.50 ppm): 0.72 (m, 4H), 0.78 (m, 2H), 2.71 (t, J = 7.4 Hz, 2H), |
| | 2.97 (s, 3H), 3.34 (m, 2H), 3.54 (d, J = 6 Hz, 2H), 4.38 (s, 1H), 4.74 (m, |
| | 2H), 7.34 (m, 8H), 7.47 (m, 2H), 7.78 (m, 1H), 7.85 (m, 1H), 8.74 (s, |
| | 1H), Mass spectrum: ES m/z = 574 (MH⁺, base peak) |
| 29 | ¹H NMR spectrum (400 MHz; (δ in ppm); (d₆-DMSO); referenced at |
| | 2.50 ppm): 0.38 (m, 2H), 0.47 (m, 2H), 2.72 (t, J = 7.4 Hz, 2H), |
| | 2.97 (s, 3H), 3.33 (m, 6H); 4.39 (s, 1H); 4.54 (t, J = 4.5 Hz, 1H); |
| | 4.75 (quintuplet, J = 6.6 Hz, 1H); 7.34 (m, 8H); from 7.53 to 7.46 (m, 2H); |
| | 7.77 (m, 1H); 7.83 (double triplet, J = 6.9 and 1.8 Hz, 1H); 8.49 (t, J = 5.9 Hz, |
| | 1H); Mass spectrum: ES m/z = 588 (MH⁺, base peak) |
| 30 | ¹H NMR spectrum (400 MHz; (δ in ppm); (d₆-DMSO); referenced at |
| | 2.50 ppm): 2.74 (t, J = 7 Hz, 2H), 3.01 (s, 3H), 3.38 (m, 2H), 3.63 (m, |
| | 1H), 4.20 (m, 1H), 4.41 (s, 1H), 4.73 (quintuplet, J = 6.6 Hz, 1H), |
| | 6.52 (d, J = 6.3 Hz, 1H), 7.35 (m, 8H), 7.45 (double triplet, J = 9.5 and 2 Hz, |
| | 1H), 7.67 (m, 1H), 7.69 (m, 1H), 8.88 (t, J = 5.7 Hz, 1H), Mass |
| | spectrum: ES m/z = 634 (MH⁺, base peak) |
| 31 | ¹H NMR spectrum (400 MHz; (δ in ppm); (d₆-DMSO); referenced at |
| | 2.50 ppm): 0.72 (m, 4H), 1.76 (t, J = 7 Hz, 2H), 2.74 (t, J = 7.4 Hz, |
| | 2H), 3.00 (s, 3H), 3.36 (m, 2H), 3.53 (quartet, J = 5.9 Hz, 1H), 4.33 (t, |
| | J = 5.3 Hz, 1H), 4.41 (s, 1H), 4.72 (quintuplet, J = 6.8 Hz, 1H), |
| | 7.35 (m, 8H), 7.41 (double triplet, J = 9.4 and 2 Hz, 1H), 7.63 (m, 1H), |
| | 7.66 (m, 1H), 8.75 (s, 1H); Mass spectrum: ES m/z = 606 (MH⁺, base peak) |
| 32 | ¹H NMR spectrum (400 MHz; (δ in ppm); (d₆-DMSO); referenced at |
| | 2.50 ppm): 0.72 (m, 2H), 2.74 (t, J = 7.4 Hz, 2H), 3.00 (s, 3H), |
| | 3.36 (m, 2H), 3.53 (d, J = 6 Hz, 2H), 4.41 (s, 1H), from 4.77 to 4.68 (m, |
| | 2H); from 7.44 to 7.30 (m, 9H); 7.66 (m, 1H), 7.68 (m, 1H), 8.82 (s, |
| | 1H), Mass spectrum: ES m/z = 592 (MH⁺, base peak) |
| 33 | 1H NMR spectrum (400 MHz; (δ in ppm); (d₆-DMSO); referenced at |
| | 2.50 ppm): 0.39 (m, 2H), 0.47 (m, 2H), 2.75 (t, J = 7.3 Hz, 2H); |
| | 3.00 (s, 3H), 3.37 (m, 6H), 4.42 (s, 1H), 4.51 (t, J = 5.8 Hz, 1H); |
| | 4.74 (quintuplet, J = 6.5 Hz, 1H), 7.35 (m, 8H), 7.43 (double triplet, J = 9.4 |
| | and 2 Hz, 1H), from 7.70 to 7.64 (m, 2H), 8.56 (t, J = 5.7 Hz, 1H); |
| | Mass spectrum: ES m/z = 606 (MH⁺, base peak) |
| 34 | 1H NMR spectrum (400 MHz; (δ in ppm); (d₆-DMSO); referenced at |
| | 2.50 ppm): from 1.89 to 1.71 (m, 6H); 2.79 (t, J = 7.3 Hz, 2H), 3.05 (s, |
| | 3H), 3.42 (m, 6H), 4.46 (s, 1H), 4.65 (t, J = 6 Hz, 1H), |
| | 4.78 (quintuplet, J = 6.1 Hz, 1H), 7.39 (m, 8H), 7.48 (double triplet, J = 9.4 |
| | and 2.1 Hz, 1H), from 7.74 to 7.68 (m, 2H), 8.59 (t, J = 6 Hz, 1H), |
| | Mass spectrum: ES m/z = 620 (MH⁺, base peak) |
| 35 | ¹H NMR spectrum (300 MHz; (δ in ppm); (d₆-DMSO); referenced at |
| | 2.50 ppm): 2.76 (m, 2H); 3.02 (s, 3H), from 3.24 to 3.42 (m, 4H), |
| | 3.53 (q, J = 5.7 Hz, 2H), 4.41 (s, 1H), 4.74 (t, J = 5.7 Hz, 1H), 4.80 (m, 1H), |
| | 7.31 (m, 8H), 7.87 (broad s, 1H), 8.07 (broad s, 1H), 8.20 (broad s, |
| | 1H), 8.79 (t, J = 5.7 Hz, 1H), Mass spectrum: ES m/z = 616 (MH⁺, base |
| | peak) |
| 36 | ¹H NMR spectrum (300 MHz; (δ in ppm); (d₆-DMSO); referenced at |
| | 2.50 ppm): 1.15 (d, J = 6.7 Hz, 3H), 2.76 (t, J = 7.3 Hz, 2H), 3.02 (s, |
| | 3H), from 3.31 to 3.54 (m, 4H), from 3.92 to 4.15 (m, 1H), from 4.62 |
| | to 4.90 (m, 2H), from 7.21 to 7.39 (m, 8H); 7.85 (s, 1H); 8.08 (s, 1H), |
| | 8.22 (s, 1H), 8.45 (d, J = 7.8 Hz, 1H); Mass spectrum: ES m/z = 630 (MH⁺, |
| | base peak); Optical rotation: α_{D} = +8.9° (c = 0.333, DMSO) |
| 37 | ¹H NMR spectrum (300 MHz; (δ in ppm); (d₆-DMSO); referenced at |
| | 2.50 ppm): 2.76 (t, J = 7.4 Hz, 2H), 3.02 (s, 3H), from 3.31 to 3.40 (m, |
| | 2H), from 3.42 to 3.64 (m, 4H), 3.87 to 4.07 (m, 1H), 4.41 (s, 1H), |
| | 4.67 (d, J = 6.0 Hz, 2H), 4.81 (dq, J = 6.7 and 6.5 Hz, 1H); from 7.22 |
| | to 7.39 (m, 8H), 7.85 (s, 1H), 8.08 (s, 1H), 8.24 (s, 1H); 8.38 (d, J = 8.0 Hz, |
| | 1H); Mass spectrum: ES m/z = 646 (MH⁺, base peak) |
| 38 | ¹H NMR spectrum (300 MHz; (δ in ppm); (d₆-DMSO); referenced at |
| | 2.50 ppm): 1.28 (s, 3H), 2.76 (t, J = 7.2 Hz, 2H), 3.01 (s, 3H), from |
| | 3.31 to 3.38 (m, 2H), from 3.50 to 3.72 (m, 4H), 4.42 (s, 1H), 4.71 (t, J = 6.0 Hz, |
| | 2H), 4.82 (quin, J = 6.3 Hz, 1H), from 7.23 to 7.40 (m, 8H), |
| | 7.72 (s, 1H), 7.83 (s, 1H), 8.00 (s, 1H), 8.14 (s, 1H); Mass spectrum: |
| | ES m/z = 660 (MH⁺, base peak) |
| 39 | ¹H NMR spectrum (300 MHz; (δ in ppm); (d₆-DMSO); referenced at |
| | 2.50 ppm): from 0.64 to 0.79 (m, 4H), 1.77 (t, J = 7.0 Hz, 2H), 2.76 (t, |
| | J = 7.0 Hz, 2H); 3.01 (s, 3H); 3.33 (t, J = 7.3 Hz, 2H), from 3.46 to |
| | 3.58 (m, 2H), 4.32 (t, J = 5.5 Hz, 1H), 4.41 (s, 1H), from 4.72 to |
| | 4.88 (m, 1H), from 7.23 to 7.36 (m, 8H), 7.84 (s, 1H), 8.04 (s, 1H); 8.17 (s, |
| | 1H), 8.95 (s, 1H); Mass spectrum: ES m/z = 656 (MH⁺, base peak) |
| 40 | ¹H NMR spectrum (300 MHz; (δ in ppm); (d₆-DMSO); referenced at |
| | 2.50 ppm): from 1.36 to 1.92 (m, 6H), from 2.64 to 2.84 (m, 2H), |
| | 3.02 (s, 3H), from 3.30 to 3.39 (m, 2H), from 3.94 to 4.19 (m, 2H), 4.42 (s, |
| | 1H), 4.69 (d, J = 3.4 Hz, 1H), 4.81 (quin, J = 6.6 Hz, 1H); from 7.17 to |
| | 7.42 (m, 8H); 7.84 (s, 1H), 8.08 (s, 1H), 8.26 (s, 1H), 8.37 (d, J = 7.0 Hz, |
| | 1H), Mass spectrum: ES m/z = 656 (MH⁺, base peak) |
| 41 | ¹H NMR spectrum (300 MHz; (δ in ppm); (d₆-DMSO); referenced at |
| | 2.50 ppm): from 1.40 to 1.59 (m, 2H), from 1.59 to 1.77 (m, 2H), from |
| | 1.77 to 1.94 (m, 1H), from 1.95 to 2.12 (m, 1H), 2.76 (t, J = 6.9 Hz, |
| | 2H), 3.02 (s, 3H), from 3.32 to 3.39 (m, 2H), from 3.93 to 4.08 (m, |
| | 2H), 4.41 (s, 1H), from 4.68 to 4.91 (m, 2H), from 7.23 to 7.38 (m, |
| | 8H), 7.86 (s, 1H), 8.07 (s, 1H), 8.21 (s, 1H), 8.54 (d, J = 6.8 Hz, 1H), |
| | Mass spectrum: ES m/z = 656 (MH⁺, base peak) |
| 42 | ¹H NMR spectrum (400 MHz; (δ in ppm); (d₆-DMSO); referenced at |
| | 2.50 ppm): from 1.43 to 1.87 (m, 6H), from 2.68 to 2.78 (m, 2H), |
| | 2.99 (s, 3H), 3.35 (t, J = 7.3 Hz, 2H), 4.04 (br. s., 2H), 4.41 (s, 1H), 4.68 (d, |
| | J = 3.2 Hz, 1H), from 4.70 to 4.77 (m, 1H), from 7.27 to 7.40 (m, 8H), |
| | 7.41 (t, J = 2.0 Hz, 1H), 7.66 (t, J = 2 Hz, 1H), from 7.69 to 7.75 (m, |
| | 1H), 8.10 (d, J = 7.6 Hz, 1H), Mass spectrum: ES m/z = 606 (MH⁺, base |
| | peak) |
| 43 | ¹H NMR spectrum (400 MHz; (δ in ppm); (d₆-DMSO); referenced at |
| | 2.50 ppm): from 1.44 to 1.85 (m, 6H), from 2.69 to 2.77 (m, 2H), |
| | 2.99 (s, 3H), 3.35 (masked m, 2H), 4.03 (br. s., 2H), 4.41 (s, 1H), from 4.68 |
| | to 4.79 (m, 2H), from 7.27 to 7.41 (m, 8H), 7.42 (t, J = 2 Hz, 1H), |
| | 7.67 (br. s., 1H), 7.73 (d, J = 9.5 Hz, 1H), 8.16 (d, J = 7.6 Hz, 1H), Mass |
| | spectrum: ES m/z = 606 (MH⁺, base peak); Optical rotation: α_{D} = −16.8° (c = 0.456, |
| | MeOH) |
| 44 | ¹H NMR spectrum (400 MHz; (δ in ppm); (d₆-DMSO); referenced at |
| | 2.50 ppm): from 1.42 to 1.86 (m, 6H), from 2.66 to 2.77 (m, 2H), |
| | 3.00 (s, 3H), 3.15 (masked m, 2H), 4.04 (br. s., 2H), 4.41 (s, 1H), from 4.68 |
| | to 4.79 (m, 2H), from 7.27 to 7.45 (m, 9H), 7.67 (s, 1H), 7.73 (d, J = 9.0 Hz, |
| | 1H), 8.17 (d, J = 7.3 Hz, 1H); Mass spectrum: ES m/z = 606 (MH⁺, |
| | base peak); Optical rotation: α_{D} = +18.1° (c = 0.473, MeOH) |
| 45 | ¹H NMR spectrum (400 MHz; (δ in ppm); (d₆-DMSO); referenced at |
| | 2.50 ppm): from 1.39 to 2.10 (m, 6H); 2.73 (t, J = 7.0 Hz, 2H), 2.99 (s, |
| | 3H), 3.35 (t, J = 7.0 Hz, 2H), from 3.92 to 4.06 (m, 2H), 4.40 (s, 1H), |
| | from 4.66 to 4.74 (m, 1H), 4.75 (d, J = 4.0 Hz, 1H), from 7.28 to |
| | 7.39 (m, 8H), 7.41 (d, J = 9.3 Hz, 1H), 7.65 (s, 1H), 7.69 (d, J = 9.3 Hz, |
| | 1H), 8.34 (d, J = 6.0 Hz, 1H), Mass spectrum: ES m/z = 606 (MH⁺, base |
| | peak); Elemental analysis: Calculated: C: 57.43% H: 4.99% N: 6.93% |
| | S: 5.29%; Measured: C: 57.32% H: 5.18% N: 6.64% S: 5.08% |
| 46 | ¹H NMR spectrum (400 MHz; (δ in ppm); (d₆-DMSO); referenced at |
| | 2.50 ppm): 1.13 (d, J = 6.9 Hz, 3H), 2.71 (t, J = 7.0 Hz, 2H), 2.96 (s, |
| | 3H), from 3.31 to 3.49 (m, 4H), 3.81 (s, 3H), from 3.95 to 4.07 (m, |
| | 1H), 4.39 (s, 1H), from 4.67 to 4.76 (m, 2H), 7.01 (t, J = 2.2 Hz, 1H), |
| | from 7.28 to 7.38 (m, 9H), 7.39 to 7.41 (m, 1H), 8.12 (d, J = 7.8 Hz, |
| | 1H), Mass spectrum: ES m/z = 592 (MH⁺, base peak); Elemental |
| | analysis: Calculated: C: 56.76% H: 5.27% N: 7.09% S: 5.41%; |
| | Measured: C: 56.78% H: 5.36% N: 7.05% S: 4.98%; Optical rotation: |
| | α_{D} = +5.2° (c = 0.361, DMSO) |
| 47 | ¹H NMR spectrum (400 MHz; (δ in ppm); (d₆-DMSO); referenced at |
| | 2.50 ppm): 2.73 (t, J = 6.9 Hz, 2H), 2.99 (s, 3H), 3.30 (masked m, 2H), |
| | 3.54 (dd, J = 9.3 and 2.5 Hz, 1H), 3.64 (dd, J = 9.3 and 2.5 Hz, 1H), |
| | 3.91 (dd, J = 9.3 and 5.0 Hz, 1H), 4.00 (dd, J = 9.3 and 5.0 Hz, 1H), |
| | from 4.16 to 4.24 (m, 2H), 4.40 (s, 1H), 4.72 (quin, J = 6.5 Hz, 1H), |
| | 5.28 (d, J = 3.7 Hz, 1H), from 7.28 to 7.39 (m, 8H), 7.43 (dt, J = 9.4 and |
| | 2.0 Hz, 1H), 7.66 (s, 1H), 7.70 (d, J = 9.4 Hz, 1H), 8.59 (d, J = 6.4 Hz, |
| | 1H), Mass spectrum: ES m/z = 608 (MH⁺, base peak); Elemental |
| | analysis: Calculated: C: 55.27% H: 4.64% N: 6.91% S: 5.27%; |
| | Measured: C: 55.33% H: 4.66% N: 6.90% S: 5.03% |
| 48 | ¹H NMR spectrum (400 MHz; (δ in ppm); (d₆-DMSO); referenced at |
| | 2.50 ppm): from 1.40 to 2.06 (m, 6H), 2.73 (t, J = 7.2 Hz, 2H), 2.99 (s, |
| | 3H), from 3.32 to 3.40 (m, 2H), from 3.91 to 4.05 (m, 2H), 4.40 (s, |
| | 1H), from 4.67 to 4.80 (m, 2H), from 7.26 to 7.38 (m, 8H), 7.41 (dt, J = 9.5 |
| | and 2.0 Hz, 1H), 7.65 (br. s., 1H), from 7.66 to 7.73 (m, 1H), |
| | 8.34 (d, J = 6.4 Hz, 1H), Mass spectrum: ES m/z = 606 (MH⁺, base peak); |
| | Elemental analysis: Calculated: C: 57.43% H: 4.99% N: 6.93% S: |
| | 5.29%; Measured: C: 57.47% H: 5.05% N: 6.73% S: 5.09%; Optical |
| | rotation: α_{D} = −15.6° (c = 0.366, DMSO) |
| 49 | ¹H NMR spectrum (400 MHz; (δ in ppm); (d₆-DMSO); referenced at |
| | 2.50 ppm): from 1.41 to 2.06 (m, 6H), 2.73 (t, J = 7.1 Hz, 2H); 2.99 (s, |
| | 3H), from 3.32 to 3.38 (m, 2H), from 3.92 to 4.08 (m, 2H), 4.40 (s, |
| | 1H), from 4.67 to 4.79 (m, 2H), from 7.25 to 7.38 (m, 8H), 7.41 (dt, J = 9.4 |
| | and 2.0 Hz, 1H); 7.65 (br. s., 1H), from 7.66 to 7.72 (m, 1H); |
| | 8.34 (d, J = 6.6 Hz, 1H), Mass spectrum: ES m/z = 606 (MH⁺, base peak); |
| | Optical rotation: α_{D} = +20.3° (c = 0.415, DMSO) |

The compounds according to the invention have formed the subject of pharmacological assays which make it possible to determine the activity with regard to human CB1-type cannabinoid receptors. The effectiveness of the compounds of formula (I) was determined in a functional test in which the activity of the CB1 cannabinoid receptors is measured (intracellular cyclic AMP test). The test for detecting intracellular cyclic AMP in U373MG cells naturally expressing the human CB1 receptor was carried out as described in the reference: Bouaboula et al., 1995, J. Biol. Chem. 270: 13973-13980. The HTRF cAMP Dynamic Kit from CisBio was used to quantify the intracellular cyclic AMP. In this test, the IC₅₀ values are between 0.001 μM and 2 μM.

For example, compounds Nos. 5, 7, 9, 18, 21, 26, 30, 36 and 47 showed IC₅₀ values of 0.022; 0.061; 0.015; 0.006; 0.038; 0.02; 0.066; 0.016 and 0.072 μM respectively.

Other assays consisting in measuring the in vivo activity of the compounds of the invention were carried out. Their antagonist activity was shown by means of the model of hyperthermia induced by a CB cannabinoid receptor agonist (racemic CP55,940 ((1RS,3RS,4RS)-3-[2-hydroxy-4-(1,1-dimethylheptyl)phenyl]-4-(3-hydroxypropyl)cyclohexan-1-ol) at a dose of 1.25 mg/kg) in mice, according to the method described by Pertwee R. G. in Marijuana 84, Harvey D. J. eds., Oxford IRL

Press, 263-277 (1985). At time 0 min, the rectal temperature of male CD1 mice is measured before injection of the test product. At 30 minutes, a further measurement of the rectal temperature of the mice is taken and the racemic CP55,940 agonist ((1RS,3RS,4RS)-3-[2-hydroxy-4-(1,1-dimethylheptyl)phenyl]-4-(3-hydroxypropyl)cyclohexan-1-ol) (1.25 mg/kg i.p. in 10% cremophor) is administered. At 90 minutes, the rectal temperature is again measured. The results are expressed as % with respect to the control batched injected with CP 55,940 (minimum temperature) and to the carrier batch without treatment with CP55,940 (maximum temperature).

For example, compounds Nos. 9 and 25 showed a percentage of inhibition of 30% and 18% respectively at 3 mg/kg po.

Their antagonist activity was also shown by means of the model of inhibition of gastrointestinal transit induced by racemic CP55,940 ((1RS,3RS,4RS-3-[2-hydroxy-4-(1,1-dimethylheptyl)phenyl]-4-(3-hydroxypropyl)cyclohexan-1-ol) in mice, according to the method described by Rinaldi-Carmona et al., J. Pharmacol. Exp. Ther. 2004, 310, 905-914. Briefly, male CD1 mice receive the test product per os 30 minutes or 2 hours before administration of the racemic CP55,940 agonist ((1RS,3RS,4RS-3-[2-hydroxy-4-(1,1-dimethylheptyl)phenyl]-4-(3-hydroxypropyl)cyclohexan-1-ol) (0.15 mg/kg ip in 10% cremophor). After a further 30 minutes, the animals receive a charcoal bolus po. Thirty minutes later, the animals are sacrificed by euthanasia (CO₂/O₂) and the intestine is dissected. The progression of the charcoal bolus in the intestine is expressed as percentage of the total length of the intestine.

For example, compounds Nos. 9, 27, 36 and 41 showed a percentage of inhibition at 1 mg/kg po of 82, 58, 85 and 91% respectively.

Consequently, the compounds of the invention of formula (I) are in vitro and in vivo CB1-type cannabinoid receptor antagonists. Some compounds are active in vivo with regard to both the hypothermia test and the transit test, and some compounds show activities split up between the hypothermia test and the transit test.

Thus, the compounds according to the invention can be used in the treatment or prevention of diseases involving CB1 cannabinoid receptors.

For example, and without implied limitation, the compounds of formula (I) are of use as psychotropic medicaments, in particular in the treatment of psychiatric disorders, including anxiety, depression, mood disorders, insomnia, delusional disorders, obsessive disorders, psychoses in general, schizophrenia or attention deficit hyperactivity disorders (ADHD) in hyperkinetic children (MBD), and in the treatment of disorders related to the use of psychotropic substances, in particular in the case of abuse of a substance and/or of dependence on a substance, including alcohol dependence and nicotine dependence, and withdrawal disorders. The compounds of formula (I) according to the invention can be used as medicaments in the treatment of migraine, stress, illnesses of psychosomatic origin, panic attacks, epilepsy, movement disorders, in particular dyskinesias or Parkinson's disease, trembling and dystonia.

The compounds of formula (I) according to the invention can be used as medicaments for skin cancer and for protecting the skin.

The compounds of formula (I) according to the invention can also be used as medicaments in the treatment of memory disorders, cognitive disorders, in particular in the treatment of cognitive disorders related to senile dementia, to Alzheimer's disease, to schizophrenia and to neurodegenerative diseases, and in the treatment of disorders of attention or of vigilance.

Furthermore, the compounds of formula (I) can be of use as neuroprotectants, in the treatment of ischaemia, brain trauma and the treatment of neurodegenerative diseases, including Huntington's chorea or Tourette's syndrome.

The compounds of formula (I) according to the invention can be used as medicaments in the treatment of pain: neuropathic pain, acute peripheral pain, chronic pain and pain of inflammatory origin.

The compounds of formula (I) according to the invention can be used as medicaments in the treatment of disorders of appetite, of appetency (for sugars, carbohydrates, drugs, alcohol or any appetizing substance) and/or of eating behaviour, in particular in the treatment of bulimia, as well as in the treatment of type II diabetes or non-insulin-dependent diabetes and in the treatment of dyslipidaemias or the metabolic syndrome. Thus, the compounds of formula (I) according to the invention are of use in the treatment of obesity and of the risks associated with obesity, in particular the cardiovascular risks.

Furthermore, the compounds of formula (I) according to the invention can be used as medicaments in the treatment of gastrointestinal disorders, diarrhoea, ulcers, vomiting, bladder and urinary disorders, disorders of endocrine origin, cardiovascular disorders, hypotension, haemorrhagic shock, septic shock, cirrhosis, hepatic fibrosis, steatohepatitis and hepatic steatosis, whatever the etiology of these conditions: in particular, virus, alcohol, medicament, chemical, autoimmune disease, obesity, diabetes or congenital metabolic disease (haemochromatosis, alpha-1 antitrypsin deficiency, Wilson's disease, and the like), chronic liver cirrhosis, fibrosis, non-alcoholic steatohepatitis (NASH), asthma, chronic obstructive pulmonary disease, Raynaud's syndrome, glaucoma, fertility disorders, inflammatory phenomena, inflammatory diseases, diseases of the immune system, in particular autoimmune and neuroinflammatory diseases, such as rheumatoid arthritis, reactive arthritis, diseases which bring about demyelination, multiple sclerosis, infectious and viral diseases, such as encephalitis, or strokes and as medicaments for anticancer chemotherapy, in the treatment of Guillain-Barré syndrome and in the treatment of osteoporosis and sleep apnoea.

According to one of its aspects, the present invention relates to the use of a compound of formula (I), of its pharmaceutically acceptable salts and of their solvates or hydrates in the treatment of the disorders and diseases indicated above.

According to another of its aspects, the present invention relates to pharmaceutical compositions comprising, as active principle, a compound according to the invention. These pharmaceutical compositions comprise an effective dose of at least one compound according to the invention or a pharmaceutically acceptable salt of the said compound and at least one pharmaceutically acceptable excipient.

The said excipients are chosen, according to the pharmaceutical form and the method of administration desired, from the usual excipients which are known to a person skilled in the art.

In the pharmaceutical compositions of the present invention for oral, sublingual, subcutaneous, intramuscular, intravenous, topical, local, intratracheal, intranasal, transdermal or rectal administration, the active principle of formula (I) above or its salt can be administered in unit administration form, as a mixture with conventional pharmaceutical excipients, for the treatment of the abovementioned disorders or diseases.

The appropriate unit administration forms comprise oral forms, such as tablets, soft or hard gelatin capsules, powders, granules and oral solutions or suspensions, sublingual, buccal, intratracheal, intraocular or intranasal administration forms, forms for administration by inhalation, topical, transdermal, subcutaneous, intramuscular or intravenous administration forms, rectal administration forms and implants. For the topical application, the compounds according to the invention can be used in creams, gels, ointments or lotions.

By way of example, a unit administration form of a compound according to the invention in the tablet form can comprise the following components:

| | | |
|---|---|---|
| Compound according to the invention | 50.0 | mg |
| Mannitol | 223.75 | mg |
| Sodium croscarmellose | 6.0 | mg |
| Maize starch | 15.0 | mg |
| Hydroxypropylmethylcellulose | 2.25 | mg |
| Magnesium stearate | 3.0 | mg |

There may be specific cases where higher or lower dosages are appropriate: such dosages do not depart from the scope of the invention. According to the usual practice, the dosage appropriate to each patient is determined by the doctor according to the method of administration and the weight and the response of the said patient.

The present invention, according to another of its aspects, also relates to a method for the treatment of the pathologies indicated above which comprises the administration, to a patient, of an effective dose of a compound according to the invention or of one of its pharmaceutically acceptable salts.

What is claimed is:

1. A compound of formula (I): 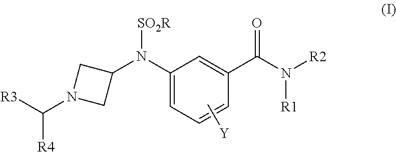
wherein:
R represents a (C₁-C₆)alkyl group or a halo(C₁-C₆)alkyl group;
R1 represents a hydrogen atom or a (C₁-C₆)alkyl group;
R2 represents a
(C₁-C₆)alkyl group substituted by one or more groups chosen from a hydroxyl group, a (C₁-C₆)alkoxy group, a hydroxy(C₁-C₆)alkyl group and optionally substituted by a halo(C₁-C₆)alkyl group;
heterocycle group optionally substituted by one or more hydroxyls, a (C₁-C₆)alkoxy group or a hydroxy(C₁-C₆)alkyl group; or
heterocycle(C₁-C₆)alkyl group optionally substituted by one or more hydroxyls;
R3 and R4 each represent a phenyl group, optionally substituted by one or more atoms or groups chosen from a hydrogen atom, a halogen, a (C₁-C₆)alkyl group, a halo(C₁-C₆)alkyl group, a (C₁-C₆)alkoxy group, a halo(C₁-C₆)alkoxy group and cyano;
Y represents a hydrogen atom, a halogen, a (C₁-C₆)alkyl group, a halo(C₁-C₆)alkyl group, a (C₁-C₆)alkoxy group, a halo(C₁-C₆)alkoxy group, a (C₁-C₆)alkylS(O)ₚ group or cyano; and
p is 0, 1 or 2;
or an acid addition salt thereof.

2. The compound of formula (I) according to claim 1, wherein:
R represents a methyl;
R3 and R4 each represent a phenyl group substituted by a chlorine atom in the para position;
Y represents a hydrogen atom or a halogen or a (C₁-C₆)alkoxy group or a halo(C₁-C₆)alkyl group;
R1 represents a hydrogen atom; and
R2 represents a
(C₁-C₆)alkyl group substituted by one or more groups chosen from a hydroxyl group, a (C₁-C₆)alkoxy group, a hydroxy(C₁-C₆)alkyl group and optionally substituted by a halo(C₁-C₆)alkyl group;
heterocycle group representing an oxetane, a tetrahydrofuran, a dioxolane or a tetrahydropyran optionally substituted by one or more hydroxyls or hydroxymethyls; or
heterocycle(C₁-C₆)alkyl group representing a tetrahydrofurylmethyl, 2,2-dimethyl-1,3-dioxolan-4-ylmethyl or 1,3-dioxolan-4-ylmethyl;
or an acid addition salt thereof.

3. The compound of formula (I) according to claim 1, wherein: 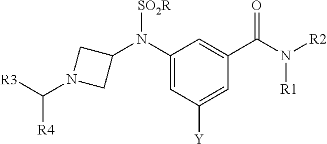
R represents a methyl;
R3 and R4 each represent a phenyl group substituted by a chlorine atom in the para position;
Y represents a hydrogen atom or a fluorine or an OMe group or a CF₃ group;
R1 represents a hydrogen atom; and
R2 represents a
(C₁-C₆)alkyl group substituted by one or more groups chosen from a hydroxyl group, an (C₁-C₆)alkoxy group, a hydroxy(C₁-C₆)alkyl group and optionally substituted by a halo(C₁-C₆)alkyl group;
an oxetane, a tetrahydrofuran, a dioxolane or a tetrahydropyran optionally substituted by one or more hydroxyls or hydroxymethyls; or
a tetrahydrofurylmethyl, 2,2-dimethyl-1,3-dioxolan-4-ylmethyl or 1,3-dioxolan-4-ylmethyl;
or an acid addition salt thereof.

4. The compound of formula (I) according to claim 1, selected from the group consisting of:
(+)-3-({1-[bis(4-chlorophenyl)methyl]azetidin-3-yl}(methanesulphonyl)amino)-N-[1-(tetrahydrofuran-2-yl)methyl]benzamide;
(−)-3-({1-[bis(4-chlorophenyl)methyl]azetidin-3-yl}(methanesulphonyl)amino)-N-[1-(tetrahydrofuran-2-yl)methyl]benzamide;
3-({1-[bis(4-chlorophenyl)methyl]azetidin-3-yl}(methanesulphonyl)amino)-N-(2,2-dimethyl-1,3-dioxolan-4-ylmethyl)benzamide;
(+)-3-({1-[bis(4-chlorophenyl)methyl]azetidin-3-yl}(methanesulphonyl)amino)-N-(2,2-dimethyl-1,3-dioxolan-4-ylmethyl)benzamide;
(−)-3-({1-[bis(4-chlorophenyl)methyl]azetidin-3-yl}(methanesulphonyl)amino)-N-(2,2-dimethyl-1,3-dioxolan-4-ylmethyl)benzamide;
3-({1-[bis(4-chlorophenyl)methyl]azetidin-3-yl}(methanesulphonyl)amino)-N-(oxetan-3-yl)benzamide;
3-({1-[bis(4-chlorophenyl)methyl]azetidin-3-yl}methanesulphonylamino)-N-(2-hydroxyethyl)benzamide hydrochloride (1:1);
(−)-3-({1-[bis(4-chlorophenyl)methyl]azetidin-3-yl}(methanesulphonyl)amino)-N-(1-hydroxyprop-2-yl)benzamide;
(+)-3-({1-[bis(4-chlorophenyl)methyl]azetidin-3-yl}(methanesulphonyl)amino)-N-(1-hydroxyprop-2-yl)benzamide;
(−)-3-({1-[bis(4-chlorophenyl)methyl]azetidin-3-yl}(methanesulphonyl)amino)-N-(2-hydroxyprop-1-yl)benzamide;
(+)-3-({1-[bis(4-chlorophenyl)methyl]azetidin-3-yl}(methanesulphonyl)amino)-N-(2-hydroxyprop-1-yl)benzamide;
3-({1-[bis(4-chlorophenyl)methyl]azetidin-3-yl}(methanesulphonyl)amino)-N-(3,3,3-trifluoro-2-hydroxyprop-1-yl)benzamide;
3-({1-[bis(4-chlorophenyl)methyl]azetidin-3-yl}(methanesulphonyl)amino)-N-(2-hydroxy-2-methylprop-1-yl)benzamide;
3-({1-[bis(4-chlorophenyl)methyl]azetidin-3-yl}(methanesulphonyl)amino)-N-(1-(hydroxymethyl)cyclopent-1-yl)benzamide;
3-({1-[bis(4-chlorophenyl)methyl]azetidin-3-yl}(methanesulphonyl)amino)-N—((S)-1-hydroxymethyl-2-methylprop-1-yl)benzamide;
3-({1-[bis(4-chlorophenyl)methyl]azetidin-3-yl}(methanesulphonyl)amino)-N-(2-hydroxy-1,1-dimethylethyl)benzamide;
3-({1-[bis(4-chlorophenyl)methyl]azetidin-3-yl}(methanesulphonyl)amino)-N-(1,3-dihydroxyprop-2-yl)benzamide;
3-({1-[bis(4-chlorophenyl)methyl]azetidin-3-yl}(methanesulphonyl)amino)-N-[1,3-dihydroxy-2-methylprop-2-yl]benzamide;
3-({1-[bis(4-chlorophenyl)methyl]azetidin-3-yl}(methanesulphonyl)amino)-N-[2-hydroxy-1,1-bis(hydroxymethyl)ethyl]benzamide;
(2R,3R,4R,5S,6R)-3-({1-[bis(4-chlorophenyl)methyl]azetidin-3-yl}(methanesulphonyl)amino)-N-[2,4,5-trihydroxy-6-(hydroxymethyl)tetrahydropyran-3-yl]benzamide;
3-({1-[bis(4-chlorophenyl)methyl]azetidin-3-yl}(methanesulphonyl)amino)-N-[1-(2-hydroxyethyl)cyclopropyl]benzamide;
(−)-3-({1-[bis(4-chlorophenyl)methyl]azetidin-3-yl}(methanesulphonyl)amino)-N-(2,3-dihydroxyprop-1-yl)benzamide;
(+)-3-({1-[bis(4-chlorophenyl)methyl]azetidin-3-yl}(methanesulphonyl)amino)-N-(2,3-dihydroxyprop-1-yl)benzamide;
3-({1-[bis(4-chlorophenyl)methyl]azetidin-3-yl}(methanesulphonyl)amino)-N-(2-methoxy-ethyl)benzamide;
(+)-3-({1-[bis(4-chlorophenyl)methyl]azetidin-3-yl}(methanesulphonyl)amino)-5-fluoro-N-(1-hydroxyprop-2-yl)benzamide;
3-({1-[bis(4-chlorophenyl)methyl]azetidin-3-yl}(methanesulphonyl)amino)-5-fluoro-N-(1,3-dihydroxyprop-2-yl)benzamide;
3-({1-[bis(4-chlorophenyl)methyl]azetidin-3-yl}(methanesulphonyl)amino)-5-fluoro-N-[1,3-dihydroxy-2-methylprop-2-yl]benzamide;
3-({1-[bis(4-chlorophenyl)methyl]azetidin-3-yl}(methanesulphonyl)amino)-N-(1-(hydroxymethyl)cycloprop-1-yl)benzamide;
3-({1-[bis(4-chlorophenyl)methyl]azetidin-3-yl}(methanesulphonyl)amino)-N-[(1-(hydroxymethyl)cycloprop-1-yl)methyl]benzamide;
3-({1-[bis(4-chlorophenyl)methyl]azetidin-3-yl}(methanesulphonyl)amino)-5-fluoro-N-(3,3,3-trifluoro-2-hydroxyprop-1-yl)benzamide;
3-({1-[bis(4-chlorophenyl)methyl]azetidin-3-yl}(methanesulphonyl)amino)-5-fluoro-N-[1-(2-hydroxyethyl)cycloprop-1-yl]benzamide;
3-({1-[bis(4-chlorophenyl)methyl]azetidin-3-yl}(methanesulphonyl)amino)-5-fluoro-N-[1-(hydroxymethyl)cycloprop-1-yl]benzamide;
3-({1-[bis(4-chlorophenyl)methyl]azetidin-3-yl}(methanesulphonyl)amino)-5-fluoro-N-[(1-(hydroxymethyl)cycloprop-1-yl)methyl]benzamide;
3-({1-[bis(4-chlorophenyl)methyl]azetidin-3-yl}(methanesulphonyl)amino)-5-fluoro-N-[(1-(hydroxymethyl)cyclobut-1-yl)methyl]benzamide;
3-({1-[bis(4-chlorophenyl)methyl]azetidin-3-yl}(methanesulphonyl)amino)-N-(2-hydroxyethyl)-5-(trifluoromethyl)benzamide;
(+)-3-({1-[bis(4-chlorophenyl)methyl]azetidin-3-yl}(methanesulphonyl)amino)-N—((S)-1-hydroxyprop-2-yl)-5-(trifluoromethyl)benzamide;
3-({1-[bis(4-chlorophenyl)methyl]azetidin-3-yl}(methanesulphonyl)amino)-N-(1,3-dihydroxyprop-2-yl)-5-(trifluoromethyl)benzamide;
3-({1-[bis(4-chlorophenyl)methyl]azetidin-3-yl}(methanesulphonyl)amino)-N-[1,3-dihydroxy-2-methylprop-2-yl]-5-(trifluoromethyl)benzamide;
3-({1-[bis(4-chlorophenyl)methyl]azetidin-3-yl}(methanesulphonyl)amino)-N-[1-(2-hydroxyethyl)cycloprop-1-yl]-5-(trifluoromethyl)benzamide;
3-({1-[bis(4-chlorophenyl)methyl]azetidin-3-yl}(methanesulphonyl)amino)-N-((1RS,2SR)-2-hydroxycyclopent-1-yl)-5-(trifluoromethyl)benzamide;
3-({1-[bis(4-chlorophenyl)methyl]azetidin-3-yl}(methanesulphonyl)amino)-N-((1SR,2SR)-2-hydroxycyclopent-1-yl)-5-(trifluoromethyl)benzamide;
3-({1-[bis(4-chlorophenyl)methyl]azetidin-3-yl}(methanesulphonyl)amino)-5-fluoro-N-((1RS,2SR)-2-hydroxycyclopent-1-yl)benzamide;
(−)-3-({1-[bis(4-chlorophenyl)methyl]azetidin-3-yl}(methanesulphonyl)amino)-5-fluoro-N-((1R*,2S*)-2-hydroxycyclopent-1-yl)benzamide;
(+)-3-({1-[bis(4-chlorophenyl)methyl]azetidin-3-yl}(methanesulphonyl)amino)-5-fluoro-N-((1S*,2R*)-2-hydroxycyclopent-1-yl)benzamide;
3-({1-[bis(4-chlorophenyl)methyl]azetidin-3-yl}(methanesulphonyl)amino)-5-fluoro-N-((1SR,2SR)-2-hydroxycyclopent-1-yl)benzamide;
(+)-3-({1-[bis(4-chlorophenyl)methyl]azetidin-3-yl}(methanesulphonyl)amino)-N—((S)-1-hydroxyprop-2-yl)-5-methoxybenzamide;
3-({1-[bis(4-chlorophenyl)methyl]azetidin-3-yl}(methanesulphonyl)amino)-5-fluoro-N-((3SR,4RS)-4-hydroxytetrahydrofuran-3-yl)benzamide;
(−)-3-({1-[bis(4-chlorophenyl)methyl]azetidin-3-yl}(methanesulphonyl)amino)-5-fluoro-N-((1S*,2S*)-2-hydroxycyclopent-1-yl)benzamide; and
(+)-3-({1-[bis(4-chlorophenyl)methyl]azetidin-3-yl}(methanesulphonyl)amino)-5-fluoro-N-((1R*,2R*)-2-hydroxycyclopent-1-yl)benzamide.

5. A pharmaceutical composition comprising the compound of formula (I) according to claim 1, or a pharmaceutically acceptable salt thereof; and one or more pharmaceutical excipients.

6. A pharmaceutical composition comprising the compound of formula (I) according to claim 2, or a pharmaceutically acceptable acid addition salt thereof; and one or more pharmaceutical excipients.

7. A pharmaceutical composition comprising the compound of formula (I) according to claim 3, or a pharmaceutically acceptable acid addition salt thereof; and one or more pharmaceutical excipients.

8. A pharmaceutical composition comprising the compound of formula (I) according to claim 4, or a pharmaceutically acceptable salt thereof; and one or more pharmaceutical excipients.

9. A method of treating or preventing a disease or disorder selected from the group consisting of psychiatric disorders, substance dependence and withdrawal, tobacco withdrawal, cognitive and attention disorders and acute and chronic neurodegenerative diseases, said method comprising administering to a patient in need thereof an effective amount of a compound of formula (I) according to claim 1 or a pharmaceutically acceptable acid addition salt thereof.

10. A method of treating or preventing a disease or disorder selected from the group consisting of metabolic disorders, appetency disorders, appetite disorders, obesity, diabetes, metabolic syndrome, dyslipidaemia and sleep apnoea, said method comprising administering to a patient in need thereof an effective amount of a compound of formula (I) according to claim 1 or a pharmaceutically acceptable acid addition salt thereof.

11. A method of treating or preventing a disease or disorder selected from the group consisting of pain, neuropathic pain and neuropathic pain induced by anticancer drugs, said method comprising administering to a patient in need thereof an effective amount of a compound of formula (I) according to claim 1 or a pharmaceutically acceptable acid addition salt thereof.

12. A method of treating or preventing a disease or disorder selected from the group consisting of gastrointestinal disorders, vomiting, ulcers, diarrhoea, bladder and urinary disorders, disorders of endocrine origin, cardiovascular disorders, hypotension, haemorrhagic shock, septic shock, liver diseases, chronic liver cirrhosis, fibrosis, non-alcoholic steatohepatitis (NASH), steatohepatitis and hepatic steatosis, said method comprising administering to a patient in need thereof an effective amount of a compound of formula (I) according to claim 1 or a pharmaceutically acceptable acid addition salt thereof.

13. A method of treating or preventing a disease or disorder selected from the group consisting of diseases of the immune system, rheumatoid arthritis, demyelination, multiple sclerosis and inflammatory diseases, said method comprising administering to a patient in need thereof an effective amount of a compound of formula (I) according to claim 1 or a pharmaceutically acceptable acid addition salt thereof.

14. A method of treating or preventing a disease or disorder selected from the group consisting of Alzheimer's disease, Parkinson's disease, schizophrenia and cognitive disorders associated with schizophrenia, with diabetes, with obesity or with metabolic syndrome, said method comprising administering to a patient in need thereof an effective amount of a compound of formula (I) according to claim 1 or a pharmaceutically acceptable acid addition salt thereof.

15. A method of treating or preventing a disease or disorder selected from the group consisting of asthma, chronic obstructive pulmonary disease, Raynaud's syndrome, glaucoma and fertility disorders, said method comprising administering to a patient in need thereof an effective amount of a compound of formula (I) according to claim 1 or a pharmaceutically acceptable acid addition salt thereof.

16. A method of treating or preventing a disease or disorder selected from the group consisting of infectious and viral diseases, strokes, Guillain-Barré syndrome, osteoporosis and sleep apnoea and for anticancer chemotherapy, said method comprising administering to a patient in need thereof an effective amount of a compound of formula (I) according to claim 1 or a pharmaceutically acceptable acid addition salt thereof.

17. A process for the preparation of a compound of formula (I) according to claim 1, wherein R, R1, R2, R3, R4 and Y are as defined in claim 1; said method comprising reacting an acid derivative 5 and an amine derivative 6: 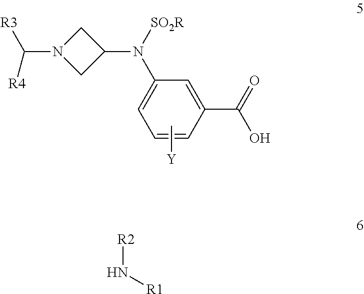
in an inert solvent, in the presence of a coupling agent and optionally in the presence of an additive which prevents racemization; optionally deprotecting the product; isolating the product and optionally converting the product to an addition salt with an acid.
